SEQUENCE LISTING (22 sequences)

SEQ ID NO: 1 - RNA, 21 nt, Artificial
  siRNA
cugucugucugcucuagcann

SEQ ID NO: 2 - RNA, 21 nt, Artificial
  siRNA
cuguuggauugauucgaaann

SEQ ID NO: 3 - DNA, 20 bp, Artificial
  primer
ctccccaccttgaatgaaga

SEQ ID NO: 4 - DNA, 20 bp, Artificial
  primer
acatagccggaacctacgtg

SEQ ID NO: 5 - DNA, 20 bp, Artificial
  primer
aactttggcattgtggaagg

SEQ ID NO: 6 - DNA, 20 bp, Artificial
  primer
acacattgggggtaggaaca

SEQ ID NO: 7 - DNA, 20 bp, Artificial
  primer
caccaagcacatttactcca

SEQ ID NO: 8 - DNA, 20 bp, Artificial
  primer
gtaaccatcacagaagccag

SEQ ID NO: 9 - DNA, 22 bp, Artificial
  primer
cgacaaatacgttgaaatgcac

SEQ ID NO: 10 - DNA, 21 bp, Artificial
  primer
cactgttgaggtatttcccag

SEQ ID NO: 11 - DNA, 20 bp, Artificial
  primer
tcaaatgatccagacatgcg

SEQ ID NO: 12 - DNA, 21 bp, Artificial
  primer
tggatgggataagggaaagtc

SEQ ID NO: 13 - DNA, 21 bp, Artificial
  primer
aagttgtgttctccagtttcc

SEQ ID NO: 14 - DNA, 20 bp, Artificial
  primer
gtatcactcagcaccatcag

SEQ ID NO: 15 - DNA, 22 bp, Artificial
  primer
ttgccaatgctacaactaaacc

SEQ ID NO: 16 - DNA, 23 bp, Artificial
  primer
gagcatagacaacaacttcttcc

SEQ ID NO: 17 - DNA, 24 bp, Artificial
  primer
catctctgatacataggcatagac

SEQ ID NO: 18 - DNA, 24 bp, Artificial
  primer
ttcagatattggtagtctcgaagg

SEQ ID NO: 19 - DNA, 21 bp, Artificial
  primer
agcactctgggcaatctcagg

SEQ ID NO: 20 - DNA, 24 bp, Artificial
  primer
aaggtctcaatgttggagatgacg

SEQ ID NO: 21 - DNA, 20 bp, Artificial
  primer
gcaaagtgaaagaggaagtg

SEQ ID NO: 22 - DNA, 22 bp, Artificial
  primer
aaggttgctcttctttacatcc

(19) United States
(12) Patent Application Publication
(10) Pub. No.: US 2011/0293750 A1
(43) Pub. Date: Dec. 1, 2011

(54) ACTIVATED WNT-BETA-CATENIN SIGNALING IN MELANOMA

(75) Inventors: Randall T. Moon, Kenmore, WA (US); Andy J. Chien, Bellevue, WA (US); David L. Rimm, New Haven, CT (US)

(73) Assignees: YALE UNIVERSITY, New Haven, CT (US); UNIVERSITY OF WASHINGTON, Seattle, WA (US)

(21) Appl. No.: 13/128,673

(22) PCT Filed: Nov. 10, 2009
(86) PCT No.: PCT/US2009/063858

§ 371 Date: Aug. 3, 2011

Related U.S. Application Data
(60) Provisional application No. 61/113,461, filed on Nov. 11, 2008.

(51) Int. Cl. A61K 31/428; G01N 33/566; C40B 30/00; A61K 38/22; A61K 31/404; A61K 33/00; A61K 38/28; A61K 38/30; C12Q 1/68; A61K 31/495

(52) U.S. Cl. 424/722; 435/6.12; 436/501; 506/7; 435/7.1; 435/7.92; 514/367; 514/255.04; 514/414; 514/5.9; 514/8.6; 514/9.6

(57) ABSTRACT

The present invention is directed to a method of determining the prospects for survival of a melanoma patient. This method involves providing a biological sample from a patient diagnosed with melanoma, determining the level of an indicator of Wnt/β-catenin activation in the sample, comparing the level of the indicator of Wnt/β-catenin activation in the sample against a standard level of the indicator of Wnt/β-catenin activation correlated to survival of melanoma, and determining a patient's prospects for survival of melanoma based on the comparison. The present invention also relates to a method of improving survival of melanoma patients, decreasing metastases in melanoma patients, decreasing proliferation of cancer cells in melanoma patients, decreasing melanoma recurrence in melanoma patients, and/or decreasing tumor size in melanoma patients. This involves selecting melanoma patients and subsequently determining and monitoring therapy based on their level of Wnt/β-catenin activation. The selected melanoma patients are treated with a dose of an activator or synergizer of Wnt/β-catenin based on the patient's level of Wnt/β-catenin, under conditions effective, respectively, to improve survival of the selected melanoma patients, to decrease metas-tases in the selected melanoma patients, to decrease proliferation of cancer cells in the selected melanoma patients, to decrease melanoma recurrence in the selected melanoma patients, and/or to decrease tumor size in the selected melanoma patients.

This application claims benefit of U.S. Provisional Patent Application Ser. No. 61/113,461, filed Nov. 11, 2008, which is hereby incorporated by reference in its entirety.

This invention was made with government support under grant number 1K08128565-01 awarded by National Institutes of Health (NIH). The government has certain rights in this invention.

### FIELD OF THE INVENTION

The present invention is directed to activated Wnt/β-catenin signaling in melanoma.

### BACKGROUND OF THE INVENTION

Malignant melanoma accounts for less than five percent of all skin cancers, yet is responsible for 80% of skin cancer deaths (Tsao et al., “Management of Cutaneous Melanoma,” N Engl J Med 351:998-1012 (2004)). The outlook for patients with metastatic melanoma remains quite bleak, with a five-year survival rate of 5-15% that has not changed significantly over decades despite intensive efforts to develop an effective therapy. While the molecular mechanisms underlying the formation and progression of melanoma remain unresolved, recent studies have implicated Wnt signal transduction pathways in melanoma biology (Weeraratna, A. T., “A Wnt-er Wonderland—the Complexity of Wnt Signaling in Melanoma,”Cancer Metastasis Rev 24:237-50 (2005)), raising the question of whether this insight can be used to develop a therapy.

Wnt genes encode a family of 19 secreted glycoproteins that act as ligands to activate receptor-mediated signaling pathways that control cell fate and differentiation, cell proliferation, and cell motility (Chien et al., “WNTS and WNT Receptors as Therapeutic Tools and Targets in Human Disease Processes,” Front Biosci 12: 448-57 (2007)). The extensively characterized Wnt/β-catenin pathway inhibits the degradation of β-catenin, leading to its accumulation in the nucleus and to regulation of target gene expression (Chien et al., “WNTS and WNT Receptors as Therapeutic Tools and Targets in Human Disease Processes,” Front Biosci 12: 448-57 (2007)). Vertebrates also have at least one other Wnt signaling pathway, often referred to as “noncanonical” Wnt signaling, which utilizes β-catenin-independent signaling mechanisms, and which in some contexts actively antagonizes β-catenin signaling (Chien et al., “WNTS and WNT Receptors as Therapeutic Tools and Targets in Human Disease Processes,” Front Biosci 12: 448-57 (2007); Weidinger et al., “When Wnts Antagonize Wnts,” J Cell Biol 162:753-5 (2003); Veeman et al., “A Second Canon. Functions and Mechanisms of Beta-Catenin-Independent Wnt Signaling,” Dev Cell 5:367-77 (2003)). Both Wnt pathways have been implicated in cancer. Specifically, mutations leading to constitutive activation of Wnt/β-catenin signaling are observed in colorectal cancer and in some kidney tumors (Rivera et al., “An X Chromosome Gene, WTX, is Commonly Inactivated in Wilms Tumor,” Science 315:642-5 (2007); Major et al., “Wilms Tumor Suppressor WTX Negatively Regulates WNT/beta-catenin Signaling,” Science 316:1043-6 (2007); Koesters et al., “Mutational Activation of the Beta-catenin Proto-oncogene is a Common Event in the Development of Wilms' Tumors,” Cancer Res 59:3880-2 (1999); Giles et al., “Caught Up in a Wnt Storm: Wnt Signaling in Cancer,” Biochim Biophys Acta 1653:1-24 (2003)), where activation of the pathway has been directly implicated in disease pathogenesis. This finding, coupled with the initial identification of vertebrate Wntl as an oncogene in a breast cancer screen (Rijsewijk et al., “The Drosophila Homolog of the Mouse Mammary Oncogene Int-1 is Identical to the Segment Polarity Gene Wingless,” Cell 50:649-57 (1987)), and with studies demonstrating the activation of this pathway in other cancers, has promoted the idea that Wnt/β-catenin signaling is oncogenic in most contexts (Chien et al., “WNTS and WNT Receptors as Therapeutic Tools and Targets in Human Disease Processes,” Front Biosci 12: 448-57 (2007)). In colorectal carcinoma, the majority of tumors exhibit constitutive activation of Wnt/β-catenin signaling through activating mutations of the adenomatous polyposis coli (APC) gene, and the presence of increased nuclear β-catenin has been shown to predict cancer progression to metastasis as well as decreased patient survival (Lugli et al., “Prognostic Significance of the Wnt Signalling Pathway Molecules APC, Beta-catenin and E-cadherin in Colorectal Cancer: A Tissue Microarray-based Analysis,” Histopathology 50:453-64 (2007); Wong et al., “Prognostic and Diagnostic Significance of Beta-catenin Nuclear Immunostaining in Colorectal Cancer,” Clin Cancer Res 10:1401-8 (2004); Miyamoto et al., “Nuclear Beta-catenin Accumulation as a Prognostic Factor in Dukes' D Human Colorectal Cancers,” Oncol Rep 12: 245-51 (2004); Cheah et al., “A Survival-Stratification Model of Human Colorectal Carcinomas with Beta-catenin and p27kip1,” Cancer 95: 2479-86 (2002)). Interestingly, while activating mutations in the β-catenin pathway are rare in melanoma (Omholt et al., “Cytoplasmic and Nuclear Accumulation of Beta-catenin is Rarely Caused by CTNNB1 Exon 3 Mutations in Cutaneous Malignant Melanoma,” Int J Cancer 92:839-42 (2001); Pollock et al., “Mutations in Exon 3 of the Beta-catenin Gene are Rare in Melanoma Cell Lines,” Melanoma Res 12:183-6 (2002); Reifenberger et al., “Molecular Genetic Analysis of Malignant Melanomas for Aberrations of the WNT Signaling Pathway Genes CTNNB1, APC, ICAT and BTRC,” Int J Cancer 100:549-56 (2002); Rubinfeld et al., “Stabilization of Beta-catenin by Genetic Defects in Melanoma Cell Lines,” Science 275:1790-2 (1997); Worm et al., “Genetic and Epigenetic Alterations of the APC Gene in Malignant Melanoma,” Oncogene 23:5215-26 (2004); Rimm et al., “Frequent Nuclear/cytoplasmic Localization of Beta-catenin Without Exon 3 Mutations in Malignant Melanoma,” Am J Pathol 154:325-9 (1999)), the noncanonical pathway, often activated by WNT5A, has been implicated in melanoma metastasis (Weeraratna et al., “Wnt5a Signaling Directly Affects Cell Motility and Invasion of Metastatic Melanoma,” Cancer Cell 1:279-88 (2002); Weeraratna et al., “Generation and Analysis of Melanoma SAGE Libraries: SAGE Advice on the Melanoma Transcriptome,” Oncogene 23: 2264-74 (2004); Da Formo et al., “WNT5A Expression Increases During Melanoma Progression and Correlates with Outcome,” Clin Cancer Res 14:5825-32 (2008); Bittner et al., “Molecular Classification of Cutaneous Malignant Melanoma by Gene Expression Profiling,” Nature 406:536-40 (2000)).

Of note, melanocytes arise from the neural crest cells, a multi-potent pool of precursors that also give rise to neuronal, glial, and cartilage lineages (Crane et al., “Neural Crest Stem and Progenitor Cells,” Annu Rev Cell Dev Biol 22:267-86 (2006)). Wnt/β-catenin signaling is necessary and sufficient to drive neural crest towards a melanocyte cell fate, in large part through direct regulation of transcriptional targets such as the homeobox gene microphthalmia transcription factor (MITF) (Vance et al., “The Transcription Network Regulating Melanocyte Development and Melanoma,” Pigment Cell Res 17:318-25 (2004); Lame et al., “Beta-catenin in the Melanocyte Lineage,” Pigment Cell Res 16:312-7 (2003); Dorsky et al., “Direct Regulation of Nacre, a Zebrafish MITF Homolog Required for Pigment Cell Formation, By the Wnt Pathway,” Genes Dev 14:158-62 (2000)). The observed presence of nuclear β-catenin in the majority of benign nevi along with the loss of nuclear β-catenin seen with melanoma progression (Bachmann et al., “Importance of P-cadherin, Beta-catenin, and Wnt5a/Frizzled for Progression of Melanocytic Tumors and Prognosis in Cutaneous Melanoma,” Clin Cancer Res 11:8606-14 (2005); Kageshita et al., “Loss of Beta-catenin Expression Associated with Disease Progression in Malignant Melanoma,” Br J Dermatol 145:210-6 (2001); Maelandsmo et al., “Reduced Beta-catenin Expression in the Cytoplasm of Advanced-stage Superficial Spreading Malignant Melanoma,” Clin Cancer Res 9:3383-8 (2003)) supports the hypothesis that activation of Wnt/β-catenin signaling is important for cellular homeostasis in this context. Consequently, the dysregulation of specific transcriptional programs in melanocytes and nevus cells through loss of Wnt/β-catenin signaling may contribute to the decreased survival seen in patients with tumors that lack nuclear β-catenin (Bachmann et al., “Importance of P-cadherin, Beta-catenin, and Wnt5a/Frizzled for Progression of Melanocytic Tumors and Prognosis in Cutaneous Melanoma,” Clin Cancer Res 11:8606-14 (2005); Kageshita et al., “Loss of Beta-catenin Expression Associated with Disease Progression in Malignant Melanoma,” Br J Dermatol 145:210-6 (2001); Maelandsmo et al., “Reduced Beta-catenin Expression in the Cytoplasm of Advanced-stage Superficial Spreading Malignant Melanoma,” Clin Cancer Res 9:3383-8 (2003)).

The present invention is directed to overcoming the deficiencies in the art. This involves techniques for obtaining quantitative and reproducible measurements of Wnt/β-catenin activation in clinical samples, whereas current methods are both subjective and limited by interobserver and intraobserver bias.

### SUMMARY OF THE INVENTION

A first aspect of the present invention relates to a method of determining the prospects for survival of a melanoma patient. This method includes providing a biological sample from a patient diagnosed with melanoma, determining the level of an indicator of Wnt/β-catenin activation in the sample, comparing the level of the indicator of Wnt/β-catenin activation in the sample against a standard level of the indicator of Wnt/β-catenin activation correlated to survival of melanoma, and determining a patient's prospects for survival of melanoma based on the comparison.

A second aspect of the present invention relates to a method of improving survival of melanoma patients, decreasing metastases in melanoma patients, decreasing proliferation of cancer cells in melanoma patients, decreasing melanoma recurrence in melanoma patients, and/or decreasing tumor size in melanoma patients. This involves selecting melanoma patients based on their level of Wnt/β-catenin. The selected melanoma patients are treated with a dose of an activator or synergizer of Wnt/β-catenin based on the patient's level of Wnt/β-catenin. Such treating is carried out under conditions effective, respectively, to improve survival of the selected melanoma patients, to decrease proliferation of cancer cells in the selected melanoma patients, to decrease melanoma recurrence in the selected melanoma patients, and/or to decrease tumor size in the selected melanoma patients.

The present invention demonstrates that in malignant melanoma elevated levels of nuclear β-catenin in both primary tumors and metastases correlate with reduced expression of a marker of proliferation and with improved survival, in contrast to colorectal cancer. The reduction in proliferation observed in vivo is recapitulated in B16 murine melanoma cells and in human melanoma cell lines cultured in vitro by either WNT3A or small molecule activators of β-catenin signaling. Consistent with these results, B16 melanoma cells expressing WNT3A also exhibit decreased tumor size and decreased metastasis when implanted into mice. Genome-wide transcriptional profiling reveals that WNT3A upregulates genes implicated in melanocyte differentiation, several of which are usually down-regulated with melanoma progression (Ryu et al., “Comprehensive Expression Profiling of Tumor Cell Lines Identifies Molecular Signatures of Melanoma Progression,” PLoS ONE 2(7):e594 (2007), which is hereby incorporated by reference in its entirety). These findings suggest that WNT3A can mediate transcriptional changes in melanoma cells in a manner reminiscent of the known role of Wnt/β-catenin signaling in normal melanocyte development, thereby altering melanoma cell fate to one that may be less proliferative and potentially less aggressive. The results may explain the observed loss of nuclear β-catenin with melanoma progression in patient tumors, which could reflect a dysregulation of cellular differentiation through loss of homeostatic Wnt/β-catenin signaling.

In the present invention, it was found that quantified levels of nuclear β-catenin correlate with improved survival from melanoma. This finding is paralleled in an established murine melanoma model, where activation of Wnt/β-catenin by either WNT3A or small molecules leads to decreased proliferation in vitro and decreased tumor size in vivo. It was then demonstrated that activation of Wnt/β-catenin signaling promotes the expression of markers of melanocyte differentiation, which correlates with the observed decrease in the expression of proliferation markers in patients with higher levels of nuclear β-catenin. Interestingly, the same genes activated by Wnt/β-catenin signaling are antagonized by WNT5A, which may be relevant to disease progression given the observed increased expression of WNT5A seen in later-stage, more aggressive melanomas (Weeraratna et al., “Wnt5a Signaling Directly Affects Cell Motility and Invasion of Metastatic Melanoma,” Cancer Cell 1:279-88 (2002); Weeraratna et al., “Generation and Analysis of Melanoma SAGE Libraries: SAGE Advice on the Melanoma Transcriptome,” Oncogene 23: 2264-74 (2004); Da Formo et al., “WNT5A Expression Increases During Melanoma Progression and Correlates with Outcome,” Clin Cancer Res 14:5825-32 (2008); Bittner et al., “Molecular Classification of Cutaneous Malignant Melanoma by Gene Expression Profiling,” Nature 406:536-40 (2000), which is hereby incorporated by reference in its entirety). Together, these studies support the hypothesis that melanoma progression is associated with a loss of Wnt/β-catenin signaling, leading to dysregulated cell fate and increased proliferation.

### BRIEF DESCRIPTION OF THE DRAWINGS

FIG. 1A-D shows levels of nuclear β-catenin predict improved survival in melanoma patients. FIG. 1A shows representative tumor cores from the tissue microarray to illustrate the localization of nuclear β-catenin by automated quantitative analysis (AQUA®) (Camp et al., “Automated Subcellular Localization and Quantification of Protein Expression in Tissue Microarrays,” Nat Med 8:1323-7 (2002), which is hereby incorporated by reference in its entirety). Tumor 1 (upper panels) is representative of tumors with less nuclear β-catenin, while tumor 2 (lower panels) is representative of tumors with higher expression of nuclear β-catenin. On the left, the orientation of the histospot used for analysis is oriented on the tumor core. The middle panels illustrate how S100 and DAPI are used to respectively identify the cytoplasmic/membranous and nuclear compartments of the tumor. Staining with β-catenin, shown in the panels on the right, is co-localized with either S100 or DAPI to generate measured values of β-catenin staining in each subcellular compartment. FIG. 1B shows nuclear β-catenin measured in primary tumors staged by Breslow depth according to AJCC criteria. Tumor depth increases from T1 to T4. Bars representing the mean and S.E.M. for tumors in each stage indicate decreasing nuclear β-catenin with increased tumor depth. Gray dots represent individual tumors, while the dotted red line indicates the average for the entire cohort. The decrease in nuclear β-catenin was significant by ANOVA followed by a post-test for linear trend. FIG. 1C shows primary tumors (n=118) stratified a priori into tertiles based on nuclear β-catenin levels (see also FIG. 6A). Patients with the highest levels of nuclear β-catenin (upper tertile) exhibit a significantly increased survival probability by Kaplan-Meier analysis compared to patients in the middle and lower tertile (log-rank test). FIG. 1D shows metastatic and recurrent tumors were separated into those with the highest nuclear β-catenin levels (upper 20%, n=46) and those with lower nuclear β-catenin levels (remaining 80%, n=179). The level of nuclear β-catenin in the upper 20% of metastatic/recurrent tumors corresponds with the levels of nuclear β-catenin seen in the upper tertile of primary tumors (FIG. 6B). Kaplan-Meier analysis showed a significantly increased survival probability in patients with the highest amount of nuclear β-catenin (Gehan-Breslow-Wilcoxon test).

FIG. 2A-F shows Wnt/β-catenin activation is associated with decreased proliferation in melanoma. FIG. 2A showing immunofluorescent staining demonstrates increased nuclear β-catenin in B16 cells expressing WNT3A, consistent with activation of the Wnt/β-catenin pathway. Cells expressing either GFP or WNT5A do not exhibit similar nuclear localization of β-catenin. FIG. 2B shows conditioned media from B16:GFP, B16:WNT3A and B16:WNT5A cells incubated with a human melanoma cell line (UACC 1273) stably transduced to express firefly luciferase under the control of a Wnt/β-catenin-responsive promoter. Media from B16:WNT3A cells activates the reporter, indicating that these cells secrete active WNT3A. No activation was seen in CM isolated from B16:GFP or B16:WNT5A cells. FIG. 2C shows proliferation of B16:GFP, B16:WNT3A, or B16:WNT5A cells measured by hematocytometer after six days of culture (black bars, left y-axis) or by MTT assay after three days of culture (white bars, right y-axis). Bars represent the average and standard deviation of three to six biological replicates. The inhibition of proliferation seen with WNT3A cells is extremely significant by ANOVA with both proliferation assays (*p<0.001). FIG. 2D shows in cell cycle analysis, B16:WNT3A cells demonstrate a decreased population in S phase and an increased population in G1 compared to B16:GFP or B16:WNT5A cells. Bars indicate the average and standard deviation of three biologic replicates, and the data shown are representative of five individual experiments, each with at least three biologic replicates per condition. The changes observed in B16:WNT3A cells are extremely significant by ANOVA (*p<0.001). FIG. 2E shows that tumor explants from B16 cells expressing WNT3A form smaller tumors than cells expressing GFP or WNT5A. Data are expressed as the mean and standard deviation from four mice for each tested cell line. The experiment shown is representative of four independent experiments with the same result, with each experiment involving at least four mice for each cell line tested. The decrease in tumor size with WNT3A was highly significant by ANOVA at 14 days post-implantation (*p=0.004). FIG. 2F shows metastases to the popliteal sentinel lymph node bed were evaluated by Firefly luciferase assay, demonstrating significantly decreased metastases in tumors expressing WNT3A. Bars represent mean and standard deviation, and the data shown is representative of four independent experiments.

FIG. 3A-F shows pharmacologic inhibition of GSK3 mimics the effects of WNT3A on proliferation. FIG. 3A-B show immunofluorescent staining of β-catenin demonstrating increased nuclear β-catenin in B16 cells treated with 10 mM lithium chloride (FIG. 3A) or 1 μM BIO (FIG. 3B) compared to control cells treated with 10 mM sodium chloride or equal volume DMSO, respectively, consistent with activation of the Wnt/β-catenin pathway by lithium and BIO. FIG. 3C-D show that quantitative PCR demonstrates increased Axin2 levels in B16 cells treated with 10 mM lithium chloride (FIG. 3C) or 1 μM BIO (FIG. 3D) compared to control cells, also consistent with activation of the Wnt/β-catenin pathway by both drugs. FIG. 3E-F show representative MTT proliferation assays demonstrate the decreased proliferation seen in B16 cells treated with 10 mM lithium chloride (FIG. 3E) or 1 μM BIO (FIG. 3F) compared to control cells. Bars represent the mean and standard deviation of three to six biologic replicates. The difference is extremely significant by unpaired two-tailed t-test (p<0.001).

FIG. 4A-C shows nuclear β-catenin in patient tumors is associated with decreased proliferation. FIG. 4A shows histograms binned by 10% increments reveal the distribution of % Ki-67 within tumors stratified into tertiles by levels of nuclear β-catenin. Note the increased number of tumors with higher % Ki-67 in the presence of lower nuclear β-catenin (lower tertile), compared to the larger number of tumors with lower % Ki-67 seen in the presence of higher nuclear β-catenin (upper tertile). The mean % Ki-67 for each tertile (shown above the histograms) increases significantly with lower expression of nuclear β-catenin (*p<0.0001 by ANOVA with post-test for linear trend). FIG. 4B shows levels of nuclear β-catenin and % Ki-67 in individual tumors were analyzed by Deming regression, revealing a slope of −1.089±0.2374, suggesting that higher levels of nuclear β-catenin are associated with decreased % Ki-67. By contrast, Deming regression comparing alpha-catenin to % Ki-67 revealed a slope that was not significantly different from zero (FIG. 7B). FIG. 4C shows primary melanomas with associated data on histologic subtype (n=110 of 118) were stratified, and the plot shows mean levels of nuclear β-catenin and % Ki-67 (+/−S.E.M.) in each category. Average levels of nuclear β-catenin and % Ki-67 for the entire cohort are shown by the red and green dashed lines, respectively. Superficial spreading melanomas and spindled/desmoplastic melanomas exhibit the highest nuclear β-catenin based on relationship to the average levels for the entire cohort. Note that tumors classified as mixed superficial spreading/nodular (SS/nod; n=14) exhibit above-average nuclear β-catenin and below-average Ki-67, and thus appear to be more similar to superficial spreading melanomas (n=59) than nodular melanomas (n=16). Likewise, amelanotic melanomas (n=8), with below-average nuclear β-catenin and above average Ki-67, appear similar to nodular melanomas, which display the same profile. Despite levels of nuclear β-catenin that are similar to nodular melanomas, acral lentiginous melanomas (ALMs; n=8) exhibit much lower relative proliferation. Spindled or desmoplastic melanomas (n=5) have relatively higher levels of nuclear β-catenin and proliferation compared to other tumor subtypes.

FIG. 5A-D shows activation of Wnt/β-catenin signaling alters cell fate in melanoma cells. FIG. 5A shows B16:GFP, B16:WNT3A or B16:WNT5A cells were isolated at equivalent confluency, spun down and photographed in a 96-well plate, demonstrating the marked difference in pigmentation seen in melanoma cells expressing WNT3A. Increased pigmentation is also seen in B16 cells treated for four days with WNT3A-conditioned L-cell media (L-WNT3A) compared to control L-cell media (L-control). FIG. 5B shows whole genome expression profiles of B16:WNT3A or B16:WNT5A cells were initially compared to gene expression in B16:GFP cells, which served as the reference sample in two-channel microarrays. Three biologic replicates were analyzed for each cell line. The heatmap illustrates the differences between the most significant regulated genes in B16:WNT3A cells compared to B16:WNT5A cells by subsequent unpaired t-test. Genes that were among the most significantly regulated in B16:WNT3A cells are listed with normalized fold-change (log(2)) compared to B16:GFP cells shown in parentheses. The most significantly regulated genes include known Wnt/β-catenin targets, genes involved in melanocyte and neural crest differentiation, and genes implicated in melanoma prognosis or therapy. FIG. 5C shows several genes were selected for validation using quantitative real-time PCR (qRT-PCR), including genes implicated in melanocyte differentiation (Met, Kit, Sox9, Mitf Si/Gp100), melanoma biology (Trpm1, Kit, Mme, Mlze), and genes that are known Wnt target genes (Axing, Met, Sox9). Genes that were upregulated in B16:WNT3A cells by transcriptional profiling are all upregulated by qRT-PCR, while genes that are downregulated in B16:WNT3A cells on the array (Mlze, Mme) are also downregulated by qRT-PCR. Genes upregulated in B16:WNT3A cells are universally downregulated in the B16:WNT5A cells, providing evidence that WNT5A can antagonize transcription of Wnt/β-catenin gene targets in melanoma cells, even in the absence of WNT3A. Data are expressed as log(2)-transformed fold-change (with standard error) compared to B16:GFP cells, and are representative of three or more experiments with similar results. FIG. 5D shows gene changes induced in B16:WNT3A cells are antagonized upon treatment with β-catenin siRNA (20 nM) compared to control siRNA (20 nM). Data are expressed as log(2)-transformed fold-change (with standard error) in cells treated with β-catenin siRNA compared to control siRNA. Note that genes upregulated in (FIG. 5C) are down-regulated with β-catenin siRNA, while genes downregulated in (FIG. 5C) are upregulated with β-catenin siRNA.

FIG. 6A shows a bar histogram binned by nuclear β-catenin levels reveals the distribution within primary tumors (n=1 18) and within the tertiles used to stratify tumors. This stratification was performed a priori based on the near-Gaussian distribution of tumors with varying levels of nuclear β-catenin.

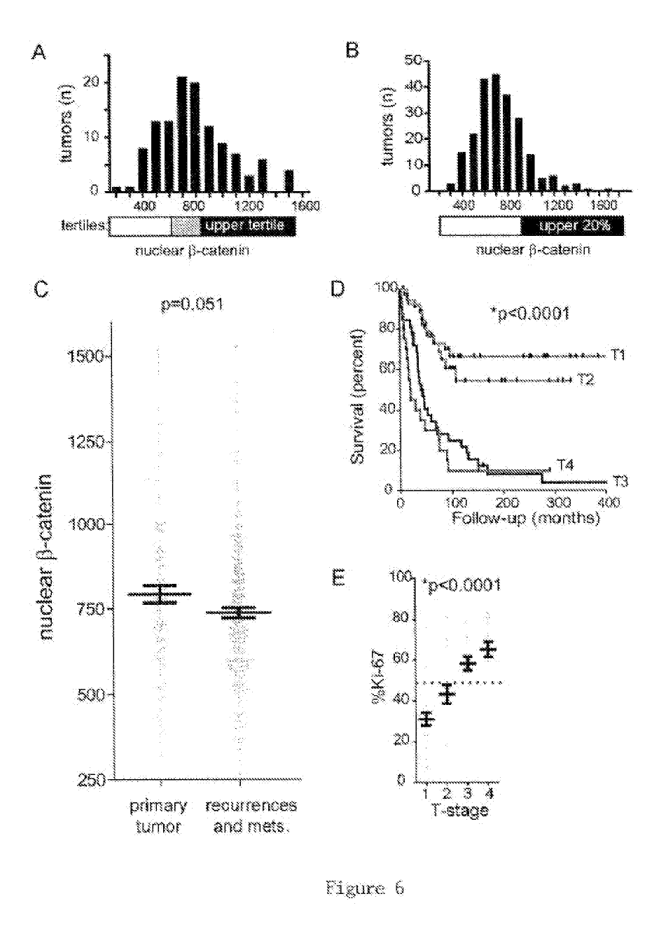

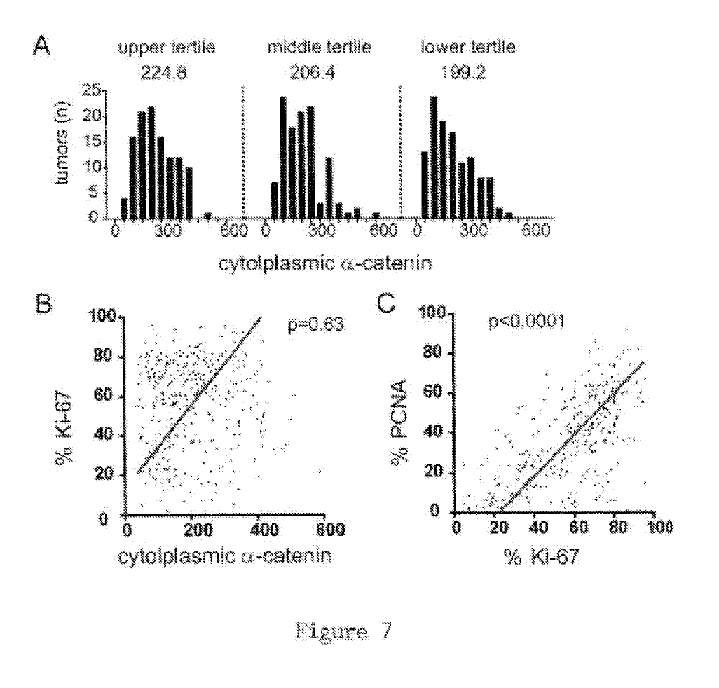

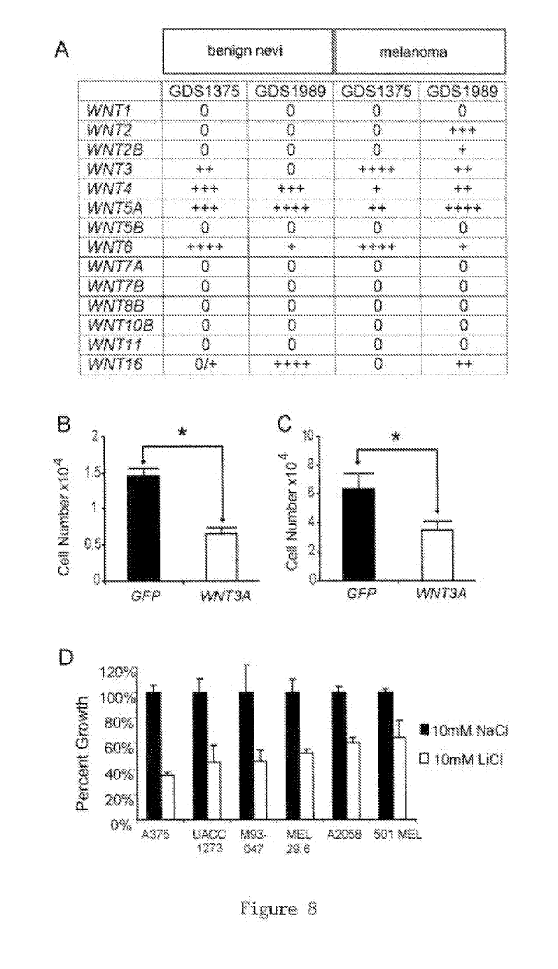

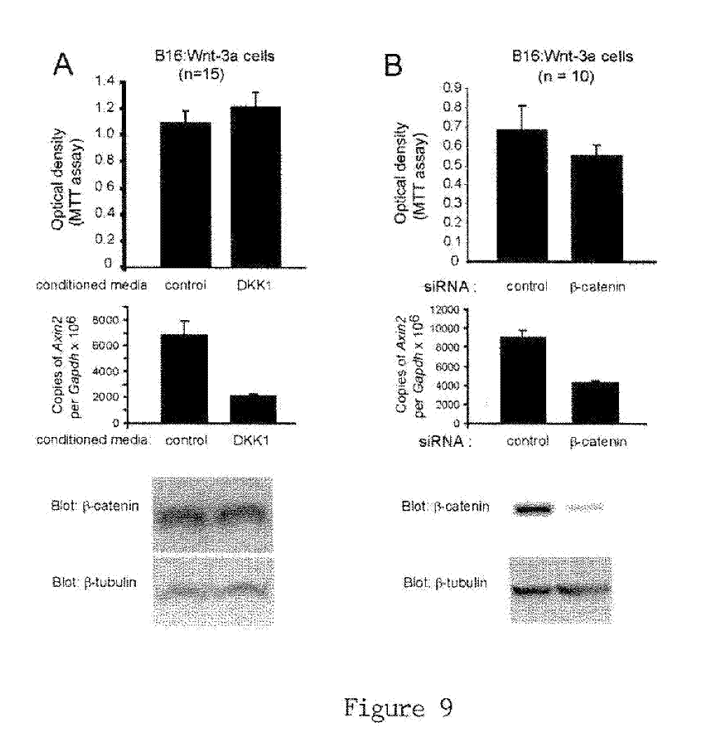

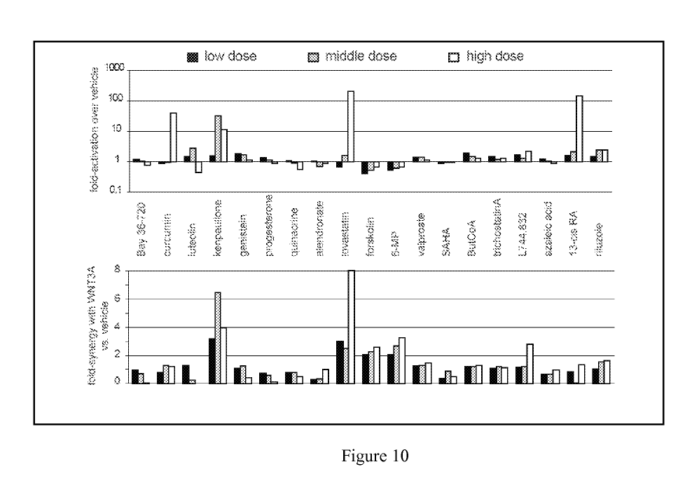

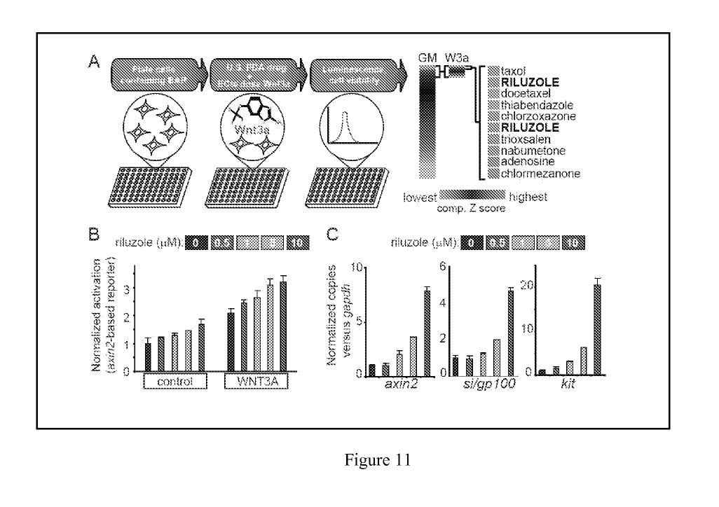

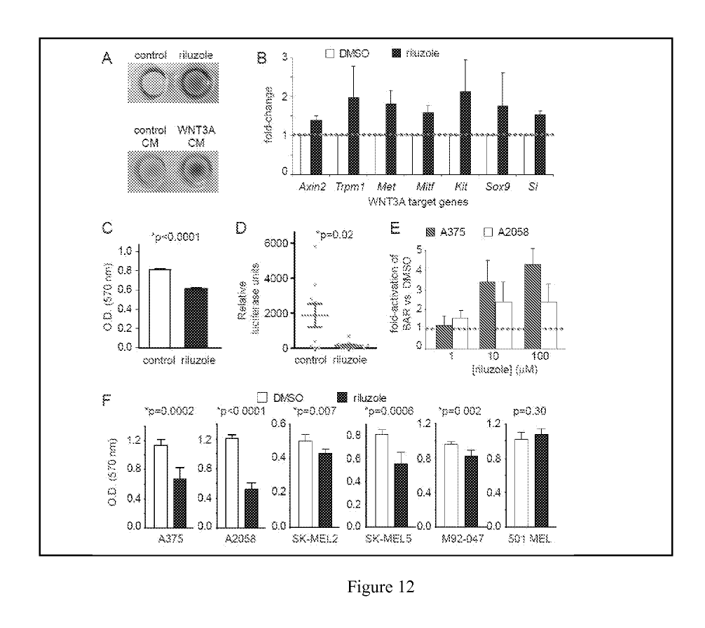

For survival analysis, FIG. 6B shows stratified nuclear β-catenin in recurrences and metastases by grouping the top 20% of tumors by nuclear β-catenin levels, which corresponds to the same levels seen in the upper tertile of primary tumors. In FIG. 6C, bars show the average and S.E.M. of nuclear β-catenin levels in primary and metastatic/recurrent melanoma tumors. Gray dots represent individual tumors. The decreased nuclear β-catenin seen in metastases/recurrences compared to primary tumors approached, but did not meet, the criteria for statistical significance based on an unpaired two-tailed t-test. In FIG. 6D, when tumors are stratified by AJCC staging criteria for tumor depth, Kaplan-Meier analysis reveals a significant survival trend (by log-rank test) based on Breslow thickness. FIG. 6E shows tumors stratified by tumor depth, where increasing Breslow thickness corresponds to increased proliferation as measured by % Ki-67. Bars represent the average and S.E.M. of individual tumors (gray dots), and the dotted red line represents the average for the entire cohort.

FIG. 7A shows bar histograms reveal the distribution of α-catenin levels (binned by AQUA® score) in patients stratified by levels of nuclear β-catenin (FIG. 6A). The difference in mean α-catenin levels (shown above the histograms) between tertiles is not significant by ANOVA followed by post-test for linear trend. FIG. 7B shows a Deming regression of % Ki-67 and levels of cytoplasmic/membranous α-catenin (in red) resulted in a line with a slope that was not significantly different from zero. FIG. 7C shows a comparison of % Ki-67 and % PCNA by Deming regression (red line) revealed a slope of 1.035±0.07615. This result suggests that within this array, % Ki-67 is a valid marker of proliferation.

FIG. 8A shows data from the NCBI Gene Expression Omnibus used to evaluate the expression of Wnt isoforms in benign nevi and melanoma tumors (see also Barrett et al., “NCBI GEO: Mining Tens of Millions of Expression Profiles—Database and Tools Update” Nucleic Acids Res 35:D760-5 (2007), which is hereby incorporated by reference in its entirety). The datasets used include GDS1375 (Talantov et al. “Novel Genes Associated with Malignant Melanoma but Not Benign Melanocytic Lesions” Clin Cancer Res October 15; 1 1(20):7234-42 (2005), which is hereby incorporated by reference in its entirety) and GDS1989 (Smith et al., “Whole-genome Expression Profiling of the Melanoma Progression Pathway Reveals Marked Molecular Differences Between Nevi/melanoma In Situ and Advanced-stage Melanomas,” Cancer Biol Ther September; 4(9):1018-29 (2005), which is hereby incorporated by reference in its entirety). The table summarizes the data from these two datasets based on the reported ‘detection call’ of the Affymetrix data used for all three datasets, and the scale indicates the percentage of samples with ‘present’ calls on the expression of the different Wnt isoforms. Scoring was as follows: 0 denotes ‘absent’ in all samples; +up to 25% of specimens have expression; ++25-50% of specimens have expression; +++50-75% of specimens have expression; ++++75-100% of specimens have expression. Few Wnt isoforms are expressed by melanoma tumors based on this transcriptional profiling, and only WNT3, WNT4, WNT5A, and WNT6 were detected in melanomas from both gene datasets. FIG. 8B-C show the human melanoma cell lines (FIG. 8B) Me1375 and (FIG. 8C) UACC 1273 were transduced with lentiviral constructs for encoding either GFP or WNT3A. Cells were counted after 3-7 days by hematocytometer and the panels shown are representative of multiple experiments with similar results. Bars represent the average and standard deviation from three biologic replicates. P-values for two-tailed t-tests were statistically significant (*p<0.05). Expression of WNT3A also led to a consistent and reproducible decrease in proliferation by MTT assay. No consistent effect on proliferation was seen with expression of WNT5A, again similar to the B16 cell lines. FIG. 8D shows human melanoma cell lines were cultured for 3-7 days in the presence of either 10 mM sodium chloride or 10 mM lithium chloride. Proliferation was measured by hematocytometer or MTT assay, and normalized to growth observed in the samples cultured in 10 mM sodium chloride. Lithium chloride inhibited proliferation in all human melanoma cell lines tested.

FIG. 9A-B shows Wnt/β-catenin inhibition does not appreciably restore proliferation to B16:WNT3A cells. In FIG. 9A, B16:WNT3A cells were treated for four days with either control conditioned media or conditioned media containing the secreted Wnt inhibitor DKK1. Proliferation by MTT assay (upper panel) was not significantly reversed. Quantitative real-time PCR (qRT-PCR) of Axin2 confirmed inhibition of Wnt/β-catenin signaling (middle panels), while immunoblotting showed no change in cellular levels of β-catenin (lower panels). In FIG. 9B, B16:WNT3A cells were transfected with either control siRNA or siRNA targeting β-catenin for three days, after which proliferation was measured by MTT assay (upper panel). Levels of Axin2 by qRT-PCR confirm inhibition of Wnt/β-catenin signaling (middle panels), while immunoblotting confirms knockdown of cellular β-catenin by the targeted siRNA. It is speculated that the differences in proliferation seen with DKK1 compared to β-catenin siRNA are related to changes in cellular adhesion seen with β-catenin siRNA that are not seen with DKK1, where levels of cellular β-catenin remain unchanged.

FIG. 10 shows A2058 melanoma cells expressing a Wnt/β-catenin-responsive reporter were treated overnight with three concentrations of various drugs identified in a high-throughput screen. Fold-activation over vehicle is shown for these drugs in the top panel, while the lower panel displays fold-synergy in the presence of WNT3A conditioned media. This panel represents a subset of compounds identified in the screen, and is meant to illustrate that small molecules can modulate Wnt signaling as either stand-alone activators (upper panel) or as synergistic agents in the presence of active Wnt signaling mediated by the presence of WNT3A conditioned media.

FIG. 11A outlines the high-throughput screen using cells with a Wnt-activated reporter (BAR). FDA-approved drugs were first sorted based on their ability to enhance Wnt signaling on their own (GM; growth media), and the top hits were then ranked for their ability to synergistically enhance Wnt signaling in the presence of added WNT3A conditioned media (W3a). The resulting ranked list of small molecules shows that riluzole (listed twice to reflect two independent preparations in the screen) represented two of the top six hits. Compounds are ranked by a modified compZ score that reflects their rank within the entire screen population. FIG. 11B shows the effects of riluzole on activation of a luciferase-based reporter comprised of the promoter sequence for AXIN2, a universal target gene of Wnt signaling. In HEK293 cells, riluzole activates this AXIN2-based reporter both on its own (control) and in the presence of WNT3A conditioned media, providing further validation that this drug activates Wnt signaling. FIG. 11C shows dose-dependent activation of Wnt target genes in B16 murine melanoma cells, again confirming that this drug can act as an activator of Wnt signaling.

FIG. 12A shows that riluzole, like WNT3A, can lead to pigmentation of B16 murine melanoma cells, which is accompanied by the upregulation of differentiation markers in FIG. 12B that were previously shown to be transcriptional targets of WNT3A in these cells. FIG. 12C shows an MTT assay demonstrating that riluzole inhibits the proliferation of B16 melanoma cells in culture (p value is based on data from more than four independent assays). FIG. 12D shows metastasis of B16 melanoma tumors injected into footpads of C57BL/6 mice. Melanoma cells stably express Firefly luciferase, allowing assay of the sentinel popliteal lymph node bed by a luciferase assay. Data are from 10 mice per group, treated with either vehicle (DMSO) or riluzole (dosed by IP injection). Tumor sizes were equivalent between the two groups. In FIG. 12E, riluzole exhibits dose-dependent activation of the Wnt reporter (BAR) in two human melanoma cell lines. FIG. 12F shows MTT assays from multiple melanoma lines treated for three days with either DMSO or riluzole. All lines exhibited a statistically-significant decrease in proliferation with riluzole with the exception of 501MEL, which interestingly already has constitutive Wnt signaling secondary to an activating mutation in β-catenin. Together, these data demonstrate that riluzole can activate Wnt signaling in melanoma cells, leading to effects similar to activation of Wnt signaling by WNT3A (Chien, A. J., et al., “Activated Wnt/beta-catenin Signaling in Melanoma is Associated with Decreased Proliferation in Patient Tumors and a Murine Melanoma Model,” Proc Natl Acad Sci USA 106: 1193-1198 (2009), which is hereby incorporated by reference in its entirety.)

### DETAILED DESCRIPTION OF THE INVENTION

A first aspect of the present invention relates to a method of determining the prospects for survival of a melanoma patient. This method includes providing a biological sample from a patient diagnosed with melanoma, determining the level of an indicator of Wnt/β-catenin activation in the sample, comparing the level of the indicator of Wnt/β-catenin activation in the sample against a standard level of the indicator of Wnt/β-catenin activation correlated to survival of melanoma, and determining a patient's prospects for survival of melanoma based on the comparison.

The method of determining the prospects for survival of a melanoma patient may be carried out in a human.

The biological sample may be selected from the group consisting of surgically-excised primary tumors and metastatic lesions.

The level of the indicator is the expression level of a gene that is a marker for activation of the Wnt/β-catenin pathway. The expression level of the marker gene for activation of the Wnt/β-catenin pathway can be determined by a variety of techniques, including immunoassays (e.g., enzyme linked immunoabsorbant assay (ELISA), radioimmunoassay (RIA), immunoradiometric assay (IRMA)), Western blotting, PCR, or immunohistochemistry (including) AQUA®. Of these, quantitative PCR is particularly useful.

Genes expressed as a result of activation of the Wnt/β-catenin pathway are numerous and well known. Such genes include, but are not limited to, the genes encoding the following proteins: β-catenin, tumor suppressor gene product adenomatous polyposis coli (APC), axin, glycogen synthase kinase (GSK)-3β, TCF/LEF transcription factors, crescent, groucho, CBP, frizzled receptor, frizzled related proteins, LRP, LRP5, LRP6, kremin, Dvl/Dsh (disheveled), dickkopf, GSK-3 binding protein (GBP), FRAT/GBP, Ebi, β-TrCP, Pinl, ICAT, E-cadherin, CKI, Lgs/BCL9, and Pygo. Of these, determination of the expression level of the axin2 gene is particularly suitable for carrying out the present invention.

Alternatively, the levels of any of the above proteins produced in the Wnt/β-catenin pathway can be used to determine the expression levels of corresponding genes which are a marker for activation of the Wnt/β-catenin. The level of protein indicators can be determined with an antibody which recognizes the protein (e.g., (β-catenin). The Wnt signalling pathway is described in Thorstensen et al., “WNT-inducible Signaling Pathway Protein 3, WISP-3, is Mutated in Microsatellite Unstable Gastrointestinal Carcinomas but Not in Endometrial Carcinomas,” Atlas Genet Cytogenet Oncol Haematol 7(2): 300-331 (2003), which is hereby incorporated by reference in its entirety. Detailed reviews of Wnt signalling and action are set out in Logan et al., “The Wnt Signaling Pathway in Development and Disease,” Annu Rev Cell Dev Biol 20:781-810 (2004); Wodarz et al., “Mechanisms of Wnt Signaling in Development,” Annu Rev Cell Dev Biol 14:59-88 (1998), which are hereby incorporated by reference in their entirety. The latter document also describes a number of assays for Wnt signalling.

A second aspect of the present invention relates to a method of improving survival of melanoma patients, decreasing metastases in melanoma patients, decreasing proliferation of cancer cells in melanoma patients, decreasing melanoma recurrence in melanoma patients, and/or decreasing tumor size in melanoma patients. This involves selecting melanoma patients based on their level of Wnt/β-catenin. The selected melanoma patients are treated with a dose of an activator or synergizer of Wnt/β-catenin based on the patient's level of Wnt/β-catenin. Such treating is carried out under conditions effective, respectively, to improve survival of the selected melanoma patients, to decrease metastases in the selected melanoma patients, to decrease proliferation of cancer cells in the selected melanoma patients, to decrease melanoma recurrence in the selected melanoma patients, and/or to decrease tumor size in the selected melanoma patients.

The treating may be carried out with an activator or a synergizer of Wnt/β-catenin. The dose of activator or synergizer administered increases the level of Wnt/β-catenin in the selected melanoma patient. This treating may include the use of varying doses in patients with higher levels of Wnt/β-catenin signaling and in those patients with lower levels of Wnt/β-catenin signaling. These doses would be tailored and adjusted based on levels of Wnt/β-catenin signaling seen in initial tumor specimens as well as levels of Wnt/β-catenin signaling monitored during the course of patient treatment.

Activators are agents that, e.g., bind to, stimulate, increase, open, activate, facilitate, enhance activation, sensitize, or up-regulate the activity of Wnt/13-catenin signaling (e.g., agonists).

Synergizers are agents which act with other agents to create an effect greater than that predicted by knowing only the separate effects of the individual agents.

The activator or synergizers can be in the form of: a nucleic acid comprising a nucleotide sequence that encodes a Wnt polypeptide, a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, a small organic molecule that inhibits the expression or activity of a Wnt or β-catenin antagonist, an antisense oligonucleotide that inhibits expression of a Wnt or β-catenin antagonist, a ribozyme that inhibits expression of a Wnt or β-catenin antagonist, an RNAi construct, siRNA, or shRNA that inhibits expression of a Wnt or β-catenin antagonist, an antibody that binds to and inhibits the activity of a Wnt or β-catenin antagonist, a nucleic acid comprising a nucleotide sequence that encodes a β-catenin polypeptide, a polypeptide comprising an amino acid sequence of a β-catenin polypeptide, a nucleic acid comprising a nucleotide sequence that encodes a Lef-1 polypeptide, a polypeptide comprising an amino acid sequence of a Lef-1 polypeptide.

Suitable activators of Wnt/β-catenin may be a glycogen synthase kinase-3 (GSK-3) inhibitors as exemplified in U.S. Pat. Nos. 6,057,117 and 6,608,063; and U.S. Patent Publication Nos. 2004/0092535 and 2004/0209878, which are hereby incorporated by reference in their entirety. These references describe ATP-competitive, selective GSK-3 inhibitors CHIR-911 and CHIR-837 (also referred to as CT-99021 and CT-98023, respectively). Chiron Corporation (Emeryville, Calif.). Other suitable GSK-3 inhibitors include riluzole, flunarizine, 6-bromoindirubin-3′-oxime (BIO), lithium ions, insulin, insulin-like growth factor (IGF-1), epidermal growth factor (EGF), CHIR-911, CHIR-837, CHIR-98014, CHIR-99021, CHIR-99030, and CHIR-98023. CHIR-911 can be formulated in 10% captisol solution for administration in vivo by intraperitoneal injection, with a half-maximal effective concentration [EC.sub.50] of 766 nM and >10,000 fold selectivity for GSK-3 (see Ring et al., “Selective Glycogen Synthase Kinase 3 Inhibitors Potentiate Insulin Activation of Glucose Transport and Utilization In Vitro and In Vivo,” Diabetes 52:588-95 (2003), which is hereby incorporated by reference in its entirety). CHIR-837 can be formulated in DMSO for in vitro use, with an EC.sub.50 of 375 nM and >5,000 fold selectivity for GSK-3 (see Cline et al., “Effects of a Novel Glycogen Synthase Kinase-3 Inhibitor on Insulin-stimulated Glucose Metabolism in Zucker Diabetic Fatty (fa/fa) Rats,” Diabetes 51:2903-10 (2002), which is hereby incorporated by reference in its entirety).

The method of improving survival of, decreasing metastases in, decreasing proliferation of cancer cells in, decreasing melanoma recurrence in, and/or decreasing tumor size in melanoma patients may also include determining the level of an indicator of Wnt/β-catenin activation following a treating step and, then, modifying the treating based on determining the level of an indicator of Wnt/β-catenin activation following the treating.

The selecting of the above-described method may also include providing a biological sample from a patient diagnosed with melanoma, determining the level of an indicator Wnt/β-catenin activation in the sample, and identifying patients as the selected melanoma patients by comparing the patients' level of the indicator of Wnt/β-catenin activation against a standard level of the indicator Wnt/β-catenin correlated to survival of melanoma patients, decreased metastases in melanoma patients, decreased proliferation of cancer cells in melanoma patients, decreased melanoma recurrence in melanoma patients, and/or decreased tumor size in melanoma patients. This procedure can be carried out in substantially the same fashion as described above.

The compounds of the present invention can be administered orally, parenterally, for example, subcutaneously, intravenously, intramuscularly, intraperitoneally, by intranasal instillation, by inhalation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may also be administered directly to the airways in the form of a dry powder. For use as a dry powder, the compounds of the present invention may be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants and co-solvents and may be filled into conventional aerosol containers that are closed by a suitable metering valve.

### EXAMPLES

The following examples are provided to illustrate embodiments of the present invention but are by no means intended to limit its scope.

### Materials and Methods for Examples 1-3

### Cell Lines

B16-F1 murine melanoma cells expressing firefly luciferase were used as the parental line for experiments described here (Murakami et al., “Expression of CXC Chemokine Receptor-4 Enhances the Pulmonary Metastatic Potential of Murine B16 Melanoma Cells,” Cancer Res 62:7328-34 (2002), which is hereby incorporated by reference in its entirety). Human melanoma UACC1273 and M93047 cell lines were a generous gift from Dr. Ashani Weeraratna (National Institute of Aging; Baltimore, Md.) (Bittner et al., “Molecular Classification of Cutaneous Malignant Melanoma by Gene Expression Profiling,” Nature 406:536-40 (2000), which is hereby incorporated by reference in its entirety). The human melanoma cell lines A375, A2058, MeI 29.6, and Me1501 were a generous gift from Cassian Yee (Fred Hutchinson Cancer Research Institute; Seattle, Wash.). Sequences for human WNT3A and WNT5A were amplified by polymerase chain reaction (PCR) and cloned into third generation lentiviral vectors derived from backbone vectors that were the generous gift of Dr. Luigi Naldini (Dull et al., “A Third-generation Lentivirus Vector with a Conditional Packaging System,” J Virol 72:8463-71 (1998), which is hereby incorporated by reference in its entirety). These lentiviral vectors contained an EF1-alpha promoter driving a bi-cistronic message encoding human Wnt isoforms plus GFP. Cells were sorted by fluorescence activated cell sorting (FACS) for GFP expression, with the goal of obtaining cells with approximately equivalent levels of GFP expression.

### Cell Culture

B16 murine melanoma cells and human melanoma lines were cultured in Dulbeccos modified Eagle's media (DMEM) supplemented with 2% Fetal Bovine Serum, and 1% antibiotic/antimycotic (Invitrogen; Grand Island, N.Y.) (Murakami et al., “Expression of CXC Chemokine Receptor-4 Enhances the Pulmonary Metastatic Potential of Murine B16 Melanoma Cells,” Cancer Res 62:7328-34 (2002), which is hereby incorporated by reference in its entirety). All cell lines were cultured in the presence of 0.02% Plasmocin (InvivoGen; San Diego, Calif.). Synthetic siRNAs (Invitrogen; Grand Island, N.Y.) were transfected into cultured cells at a final concentration of 20 nM using Lipofectamine 2000 (Invitrogen; Grand Island, N.Y.). For validation experiments using β-catenin siRNA in mouse melanoma cells, a total of 20 nM siRNA was used, consisting of an equimolar mix of the following two sequences: 1) CUGUCUGUCUGCUCUAGCA(dTdT) (SEQ ID NO: 1); and 2) CUGUUGGAUUGAUUCGAAA(dTdT) (SEQ ID NO: 2).

### Conditioned Media and Measurement of Wnt Pathway Activation Using a Reporter Assay

Conditioned media was collected from sub-confluent melanoma cell lines, and this media was tested for its ability to activate Wnt/β-catenin signaling in UACC1273 cells stably transduced with a previously described Wnt/β-catenin-responsive firefly luciferase reporter and a constitutive Renilla luciferase gene used for normalization (Major et al., “Wilms Tumor Suppressor WTX Negatively Regulates WNT/beta-catenin Signaling,” Science 316:1043-6 (2007), which is hereby incorporated by reference in its entirety). Conditioned media from B16 melanoma cells was spun down to clear cell debris and then incubated with reporter cells overnight. Activation of the Wnt/β-catenin reporter was measured using a dual luciferase reporter (DLR) assay kit (Promega; Madison, Wis.).

RNA Purification from B16 Melanoma Cells and PCR Analysis

Cells were cultured for approximately 72 hours until they reached 80-90% confluency. RNA was purified using the RNeasy kit using the manufacturer's protocol (Qiagen; Maryland, MD). cDNA was synthesized using Superscript Reverse Transcriptase (Invitrogen; Grand Island, N.Y.). Light Cycler FastStart DNA Master SYBR Green1 (Roche; Mannheim, Germany) was used for real-time PCR as previously described (Major et al., “Wilms Tumor Suppressor WTX Negatively Regulates WNT/beta-catenin Signaling,” Science 316:1043-6 (2007), which is hereby incorporated by reference in its entirety). Quantitative PCR results presented in the manuscript are representative of experiments performed on a minimum of three biologic replicates. Table 1 shows QPCR primers used in the B16 experiments.

TABLE 1
QPCR primers used in the B16 experiments.
All primers listed 5′-3′.

| Gene | Forward | Reverse | Amplicon (bp) |
|---|---|---|---|
| Axin2 | CTCCCCACCTTGAATGAAGA | ACATAGCCGGAACCTACGTG | 207 |
| | (SEQ ID NO: 3) | (SEQ ID NO: 4) | |
| GAPDH | AACTTTGGCATTGTGGAAGG | ACACATTGGGGGTAGGAACA | 232 |
| | (SEQ ID NO: 5) | (SEQ ID NO: 6) | |
| KIT | CACCAAGCACATTTACTCCA | GTAACCATCACAGAAGCCAG | 211 |
| | (SEQ ID NO: 7) | (SEQ ID NO: 8) | |
| MET | CGACAAATACGTTGAAATGCAC | CACTGTTGAGGTATTTCCCAG | 201 |
| | (SEQ ID NO: 9) | (SEQ ID NO: 10) | |
| MITF | TCAAATGATCCAGACATGCG | TGGATGGGATAAGGGAAAGTC | 208 |
| | (SEQ ID NO: 11) | (SEQ ID NO: 12) | |
| Mlze | AAGTTGTGTTCTCCAGTTTCC | GTATCACTCAGCACCATCAG | 211 |
| | (SEQ ID NO: 13) | (SEQ ID NO: 14) | |
| MME | TTGCCAATGCTACAACTAAACC | GAGCATAGACAACAACTTCTTCC | 198 |
| | (SEQ ID NO: 15) | (SEQ ID NO: 16) | |
| Si | CATCTCTGATACATAGGCATAGAC | TTCAGATATTGGTAGTCTCGAAGG | 237 |
| | (SEQ ID NO: 17) | (SEQ ID NO: 18) | |
| Sox9 | AGCACTCTGGGCAATCTCAGG | AAGGTCTCAATGTTGGAGATGACG | 189 |
| | (SEQ ID NO: 19) | (SEQ ID NO: 20) | |
| TRPM1 | GCAAAGTGAAAGAGGAAGTG | AAGGTTGCTCTTCTTTACATCC | 256 |
| | (SEQ ID NO: 21) | (SEQ ID NO: 22) | |

### Cell Proliferation Assays

For cell counts by hematocytometer, cells were seeded at a uniform density (usually between 10,000 to 25,000 cells per well) in a 12 or 24 well tissue culture plate in the appropriate media. At the end of 3-7 days, cells were trypsinized, resuspended in the appropriate media and counted. Dead cells were identified by 0.4% Trypan Blue stain and excluded from hematocytometer measurements. Cell proliferation experiments were performed with a minimum of six biologic replicates. Similar results were observed for all cell lines using the MTT assay (ATCC; Manassas, Va.), performed according to manufacturer's protocol. Cell cycle analysis was performed using DAPI-staining and flow cytometry. For experiments utilizing lithium or BIO, cells were treated for 24-72 hours prior to the MTT assay.

### Immunohistochemistry and Immunoblotting Studies

A polyclonal rabbit anti-β-catenin antibody (Sigma, Cat# C2206) was used for detection of β-catenin (1:1000 dilution for immunoblot, 1:200 dilution for immunohistochemistry). Cells were grown on 18 mm glass coverslips, for 48-72 hours, fixed using 4% paraformaldahyde, permeabilized using 0.25% Triton X-100, and then blocked with 10% goat serum. Goat anti-rabbit Alexa Fluor-568 antibody (Molecular Probes; Eugene, Oreg.) was diluted 1:1000. Cells were counterstained for nucleic acid with DAPI (Molecular Probes; Eugene, Oreg.). Cellular lysates were obtained by lysing cells on plate with a 0.1% NP-40 based buffer and analyzed by NuPage 4-12% gradient gels (Invitrogen; Grand Island, N.Y.). The antibody used to confirm WNT5A expression was obtained from Cell Signaling Technologies (Danvers, Mass.).

### Tumor Microarrays and Statistical Analysis

The tumor microarray used for the present invention is part of a larger previously published dataset (Kreizenbeck et al., “Prognostic Significance of Cadherin-based Adhesion Molecules in Cutaneous Malignant Melanoma,” Cancer Epidemiol Biomarkers Prev 17:949-58 (2008), which is hereby incorporated by reference in its entirety). For this study, tumor samples were recruited that had measured values by AQUA® for β-catenin, β-catenin and Ki67, and also data on Breslow depth at diagnosis; any samples missing any of these measurements were excluded a priori from the analysis. Target antibodies were as follows: β-catenin, mouse monoclonal clone CAT-7A4 (Zymed Laboratories) 1:150; and β-catenin, mouse monoclonal clone 14 (BD Transduction Laboratories) 1:2500. Primary antibodies were incubated at 4° C. overnight. These antibodies have been previously validated (Kreizenbeck et al., “Prognostic Significance of Cadherin-based Adhesion Molecules in Cutaneous Malignant Melanoma,” Cancer Epidemiol Biomarkers Prev 17:949-58 (2008), which is hereby incorporated by reference in its entirety). The secondary antibodies, Alexa 488-conjugated goat anti-rabbit (1:100; Molecular Probes) diluted into Envision anti-mouse (neat; DAKO) were applied for 1 h at room temperature. To visualize the nuclei, 4′,6-diamidino-2-phenylindole (1:100) was included with the secondary antibodies. Finally, a 10-min Cy5-tyramide (Perkin-Elmer Life Sciences) incubation labeled the target. Additional negative controls were obtained by omitting the target protein primary antibody. Automated quantitative analysis (AQUA®) was performed as previously described (Camp et al., “Automated Subcellular Localization and Quantification of Protein Expression in Tissue Microarrays,” Nat Med 8:1323-7 (2002), which is hereby incorporated by reference in its entirety). Target antigen expression levels were determined in an automated fashion, blinded to any a priori clinical information. Statistical analysis, including Kaplan-Meier survival probabilities, ANOVA, and t-tests, was performed using the GraphPad Prism software package (GraphPad Software; La Jolla, Calif.).

cDNA Microarrays

Agilent whole mouse genome array analysis was performed through the microarray core facility at the Huntsman Cancer Institute (Salt Lake City, Utah). Data analysis, including the t-test (Pan, W., “A Comparative Review of Statistical Methods for Discovering Differentially Expressed Genes in Replicated Microarray Experiments,” Bioinformatics 18:546-54 (2002), which is hereby incorporated by reference in its entirety) was performed using the TM4 microarray software suite, which is freely available online (Saeed et al., “TM4: A Free, Open-source System for Microarray Data Management and Analysis,” Biotechniques 34:374-8 (2003), which is hereby incorporated by reference in its entirety). Two-channel hybridizations were performed with labeled cDNA isolated from three biologic replicates each for cells expressing either WNT3A or WNT5A, using cDNA from GFP-expressing cells as the reference sample. These studies revealed gene sets regulated in both WNT3A and WNT5A cells.

### In Vivo Tumor Inoculation and Measurements of Lymph Node Metastasis

Footpad injections of transduced B16 melanoma cells and measurement of popliteal lymph node and lung metastasis was performed as previously described (Murakami et al., “Expression of CXC Chemokine Receptor-4 Enhances the Pulmonary Metastatic Potential of Murine B16 Melanoma Cells,” Cancer Res 62:7328-34 (2002), which is hereby incorporated by reference in its entirety). All animal studies were performed using IACUC protocols approved by institutional review boards.

### Example 1

### Levels of Nuclear β-Catenin Correlate with Improved Patient Survival

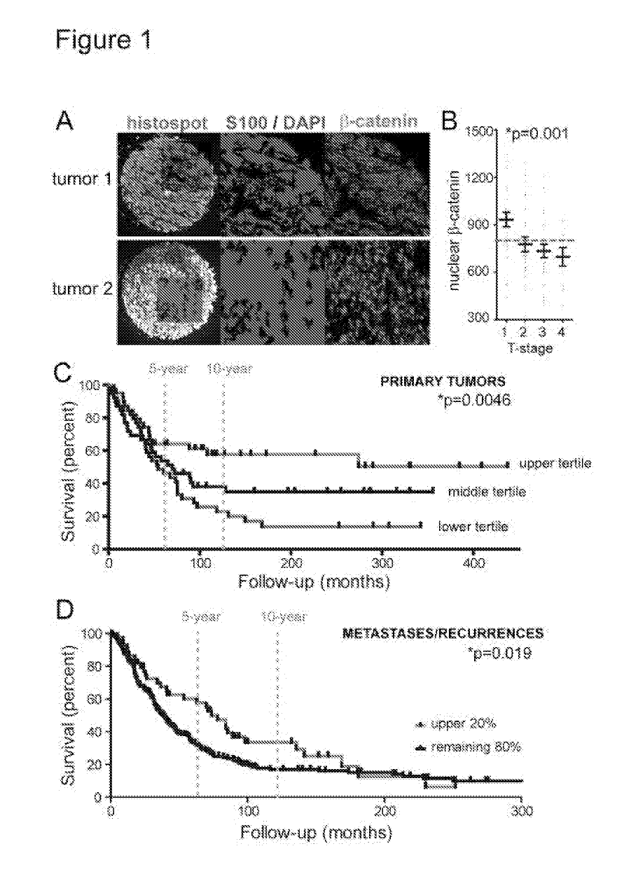

In a tissue microarray composed of 343 melanoma tumor cores (118 primary tumors plus 225 recurrences/metastases), immunohistochemical staining was utilized followed by automated quantitative analysis (AQUA®) to measure levels of nuclear β-catenin. FIG. 1A shows representative immunofluorescent staining from two different tumor cores that were measured by AQUA® (Camp et al., “Automated Subcellular Localization and Quantification of Protein Expression in Tissue Microarrays,” Nat Med 8:1323-7 (2002), which is hereby incorporated by reference in its entirety). The tumor mask was defined by S100 staining, and labeling with 4′,6-diamidino-2-phenylindole (DAPI) was used to define nuclei. This method allows for clear distinction between nuclear and cytoplasmic/membranous β-catenin. Nuclear β-catenin is higher in primary tumors compared to metastases (bordering on statistical significance by non-parametric t-test), suggesting that melanoma progression is associated with loss of Wnt/β-catenin signaling (FIG. 6C). Having tumor depth measurements (Breslow thickness) for 113 primary tumors in the array cohort, this sub-group of patients was analyzed based on the Breslow thickness stratification used in the 2002 American Joint Committee on Cancer (AJCC) melanoma staging criteria (Thompson, J. A., “The Revised American Joint Committee on Cancer Staging System for Melanoma,” Semin Oncol 29:361-9 (2002), which is hereby incorporated by reference in its entirety). Survival analysis correlated well with AJCC tumor staging by Breslow thickness (FIG. 6D). With increasing tumor thickness, a significant decrease in the levels of nuclear β-catenin (FIG. 1B) was observed, further supporting the idea that Wnt/β-catenin signaling is lost with melanoma progression.

Survival probabilities were derived using Kaplan-Meier analysis after stratifying primary tumors into tertiles based on nuclear β-catenin expression (FIG. 6A), with the upper tertile corresponding to tumors in the highest third of nuclear β-catenin, and the lower tertile corresponding to tumors with the lowest third of nuclear β-catenin. In this first use of AQUA®-scored tumor samples to correlate different levels of nuclear β-catenin with survival, it was found that higher expression of nuclear β-catenin in primary tumors predicts increased survival (FIG. 1C). Additionally, metastases and recurrences with the highest levels of nuclear β-catenin (FIG. 6B) also exhibit an increased survival probability (FIG. 1D). These findings provide the first survival analysis in metastatic melanoma based on levels of nuclear β-catenin, and more importantly suggest that even in advanced disease, activation of Wnt/β-catenin signaling still provides a survival benefit.

### Example 2

### Activation of Wnt/β-Catenin Signaling is Correlated with Decreased Proliferation of Melanoma Cells

It was next investigated whether Wnts elicit changes in melanoma cells cultured in vitro that might be consistent with the clinical observations. Since melanoma tumors express WNT3A (FIG. 8A), which has a pivotal role in the regulation of melanocyte biology (Dorsky et al., “Direct Regulation of Nacre, a Zebrafish MITF Homolog Required for Pigment Cell Formation, By the Wnt Pathway,” Genes Dev 14:158-62 (2000); Fang et al., “Defining the Conditions for the Generation of Melanocytes from Human Embryonic Stem Cells,” Stem Cells 24:1668-77 (2006), which are hereby incorporated by reference in their entirety), as well as WNT5A, which is elevated in melanoma metastases (Weeraratna et al., “Wnt5a Signaling Directly Affects Cell Motility and Invasion of Metastatic Melanoma,” Cancer Cell 1:279-88 (2002); Weeraratna et al., “Generation and Analysis of Melanoma SAGE Libraries: SAGE Advice on the Melanoma Transcriptome,” Oncogene 23: 2264-74 (2004); Da Formo et al., “WNT5A Expression Increases During Melanoma Progression and Correlates with Outcome,” Clin Cancer Res 14:5825-32 (2008); Bittner et al., “Molecular Classification of Cutaneous Malignant Melanoma by Gene Expression Profiling,” Nature 406:536-40 (2000), which are hereby incorporated by reference in their entirety), B16-F1 mouse melanoma cells were transduced with lentivirus constructs encoding WNT3A, WNT5A, or a GFP control. The subsequent cell lines are henceforth referred to as B16:GFP, B16:WNT3A, and B16:WNT5A.

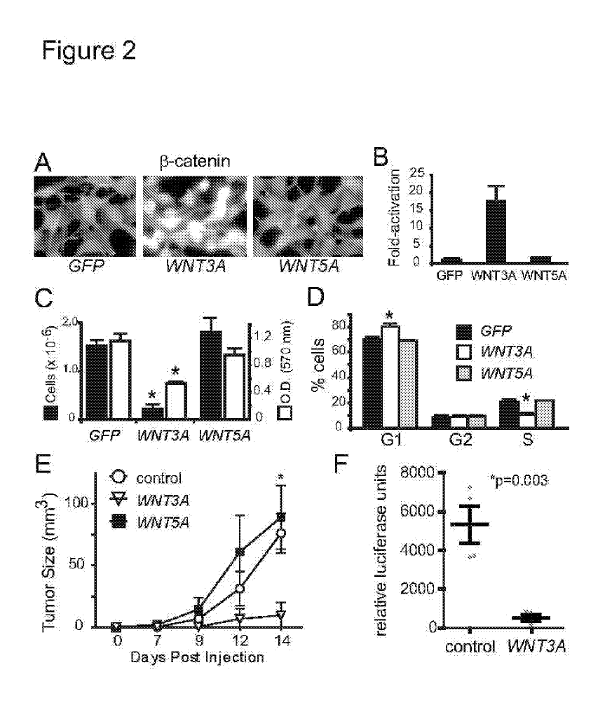

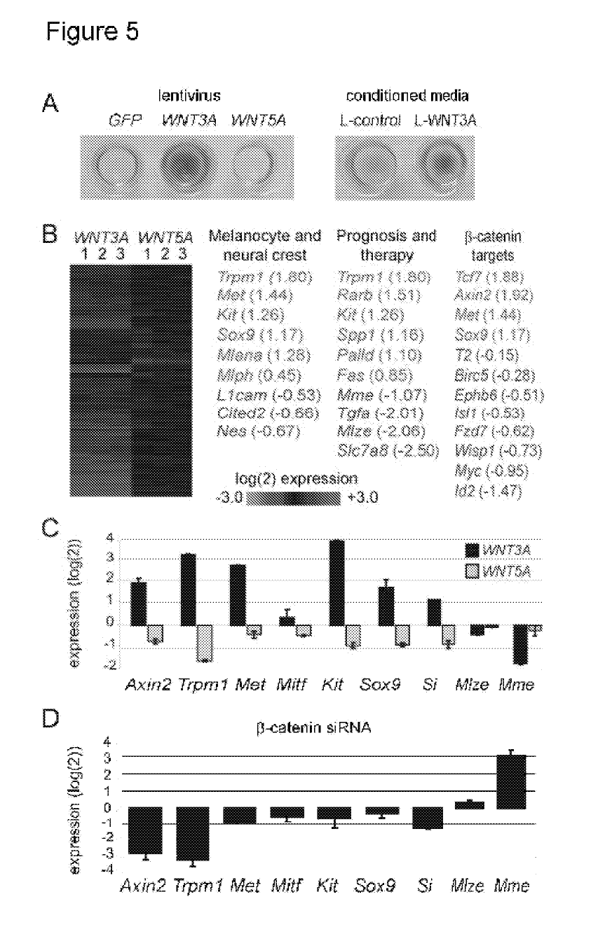

Scoring cells for nuclear accumulation of β-catenin revealed that only B16:WNT3A cells, and not B16:WNT5A or B16:GFP cells, exhibit elevated β-catenin (FIG. 2A). Expression of WNT5A was confirmed by immunoblotting. As a positive control, it was shown that conditioned media (CM) from B16:WNT3A cells, but not B16:WNT5A cells or B16:GFP, activates a β-catenin-responsive reporter in UACC1273 melanoma cells (FIG. 2B), confirming that B16:WNT3A cells secrete active WNT3A. It was then shown that B16:WNT3A cells exhibit marked upregulation of the β-catenin target gene Axin2 (Jho et al., “Wnt/beta-catenin/Tcf Signaling Induces the Transcription of Axin2, a Negative Regulator of the Signaling Pathway,” Mol Cell Biol 22:1172-83 (2002), which is hereby incorporated by reference in its entirety), compared to B16:GFP and B16:WNT5A cells (FIG. 5B).

In vitro cell proliferation studies using the MTT cell proliferation assay or manual cell counts showed that B16:WNT3A cells exhibit significantly decreased proliferation compared B16:GFP or B16:WNT5A cells (FIG. 2C). This finding was paralleled in human cell lines (FIG. 8B-C). Cell cycle analysis revealed that B16:WNT3A cells exhibit an increased population in G1, with a decreased population in S phase, compared to either B16:GFP and B16:WNT5A cells (FIG. 2D). Furthermore, in vivo tumor explants of B16:WNT3A cells exhibited decreased tumor growth (FIG. 2E) compared to either B16:GFP and B16:WNT5A cells, along with decreased metastasis (FIG. 2F). No differences in apoptosis were seen in tumor explants by TUNEL staining. Together, these data suggest that activation of Wnt/β-catenin signaling correlates with decreased proliferation of melanoma cells.

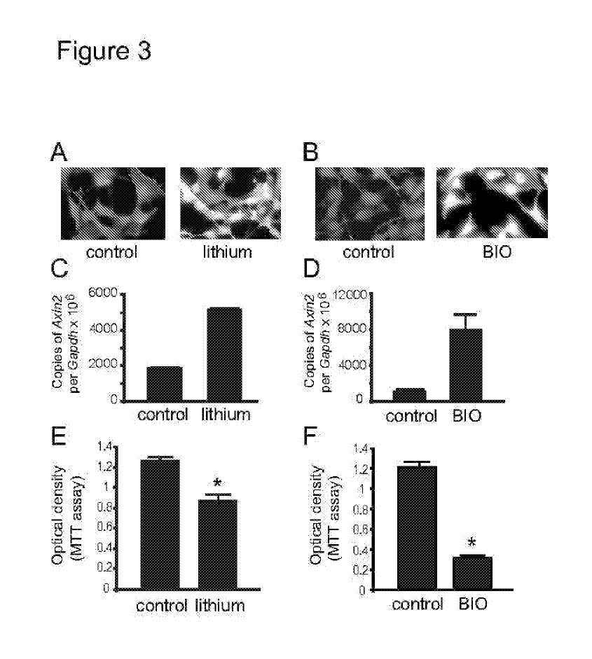

Activation of Wnt/β-catenin signaling by the GSK-3 inhibitors lithium chloride (LiCl) or 6-bromoindirubin-3′-oxime (BIO) also results in decreased proliferation of cultured B16 (FIG. 3) and human melanoma cells (FIG. 8D), supporting the hypothesis that decreased proliferation in these cells is due to activation of Wnt/β-catenin signaling. The observation that this decreased proliferation cannot be appreciably rescued by either the soluble antagonist Dickkopf-1 (DKK1) or by β-catenin-targeted siRNA (FIG. 9) suggests that this cellular change may reflect a commitment to an altered cell fate, which would be consistent with Wnt/β-catenin signaling regulating cell fate in other contexts (Moon et al., “WNT and Beta-catenin Signalling: Diseases and Therapies,” Nat Rev Genet. 5:691-701 (2004), which is hereby incorporated by reference in its entirety).

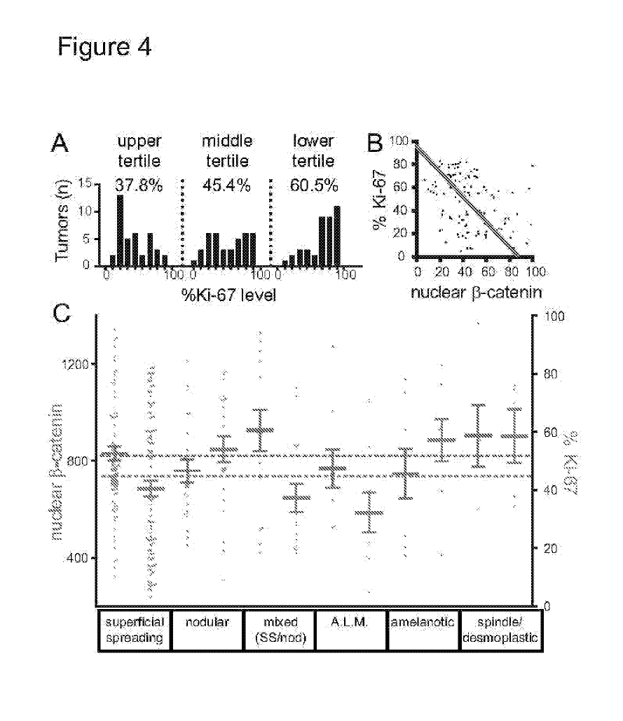

To address whether the reduced in vitro proliferation exhibited by melanoma cells with elevated β-catenin signaling reflects what is seen in patient tumors demonstrating elevated β-catenin, the expression of the cellular proliferative marker Ki-67 (% Ki-67) was analyzed in the tissue microarray cohort. Strikingly, distribution histograms of % Ki-67 staining in primary tumors stratified by expression of nuclear β-catenin show a statistically significant shift towards increased proliferation (elevated % Ki-67 staining) in the groups with lower nuclear β-catenin (FIG. 4A). There is no significant difference in the distribution of β-catenin within these tertiles (FIG. 7A). As validation for the use of % Ki-67 as a robust marker of tumor proliferation, significantly increased % Ki-67 with increasing tumor depth (FIG. 6E) was found, and also found that Ki-67 exhibited similar results within tumors to another proliferative marker, PCNA (FIG. 7C). A Deming regression of nuclear β-catenin and % Ki-67 within primary tumors is shown in FIG. 4B (slope=−1.089±0.2374; p<0.0001). By contrast, there is no relationship between expression of alpha-catenin and % Ki-67 staining (FIG. 7B). Together, the data from experimental melanoma models and from patient tumors support a model in which activation of Wnt/β-catenin signaling is associated with decreased proliferation.

These results led to the prediction that if melanomas were sorted by histologic subtype, then earlier superficial spreading melanomas in the radial growth phase would exhibit higher levels of nuclear β-catenin and lower levels of % Ki-67 compared to later-stage nodular melanomas undergoing vertical growth phase. Indeed, data from primary tumors with histologic data (n=110 of 118 tumors) support this prediction (FIG. 4C). Additionally, the relationship between nuclear β-catenin and % Ki-67 appears to be different with acral lentiginous melanomas (ALMs) and with desmoplastic/spindle cell melanoma (FIG. 4C). The hypothesis that ALMs and desmoplastic/spindle cell melanomas exhibit a distinct biology compared to more common melanomas is currently based on both histologic and clinical features, and is further supported by the comparisons of the association between nuclear β-catenin and proliferation within different melanoma subtypes.

### Example 3

### Activation of Wnt/β-Catenin Signaling Upregulates Markers of Melanocyte Differentiation

It was noted that activation of Wnt/β-catenin signaling in B16-F1 melanoma cells by either lentiviral transduction of WNT3A or by treatment of cells with conditioned media containing WNT3A results in increased pigmentation which is not seen in cells transduced with lentivirus encoding GFP or WNT5A (FIG. 5A, left panel), or cells treated with control conditioned media (FIG. 5A, right panel). These phenotypic changes, coupled with the observed decreased proliferation and altered cell cycle profile (FIGS. 2C and 2D) led us to hypothesize that activation of Wnt/β-catenin signaling leads to changes in cell fate.

To test a prediction of this hypothesis, a genome-wide transcriptional profiling was performed to determine whether expression of WNT3A in lentiviral transduced B16-F1 cells alters the expression of genes reflecting cell fates. Using gene expression in B16:GFP cells as a reference, profiles of genes regulated in B16:WNT3A and B16:WNT5A cells were first established, and then further identified genes that exhibited the highest variance between these two groups to focus on genes regulated by WNT3A. Among the most highly significant genes elevated by WNT3A (FIG. 5B) are Axin2 (Jho et al., “Wnt/beta-catenin/Tcf Signaling Induces the Transcription of Axin2, a Negative Regulator of the Signaling Pathway,” Mol Cell Biol 22:1172-83 (2002), which is hereby incorporated by reference in its entirety) and Tcf7 (Roose et al., “Synergy Between Tumor Suppressor APC and The Beta-catenin-Tcf4 Target Tcfl,” Science 285:1923-6 (1999), which is hereby incorporated by reference in its entirety), which are direct targets of Wnt/β-catenin signaling; Mme and Mlze, downregulated genes previously linked to melanoma progression (Watabe et al., “Structure, Expression and Chromosome Mapping of MLZE, a Novel Gene Which is Preferentially Expressed in Metastatic Melanoma Cells,” Jpn J Cancer Res 92:140-51 (2001); Bilalovic et al., “CD10 Protein Expression in Tumor and Stromal Cells of Malignant Melanoma is Associated with Tumor Progression,” Mod Pathol 17:1251-8 (2004), which are hereby incorporated by reference in their entirety); Mitf, linked to pigment cell fate; and Trpm1, Met, Sox9, and Kit, which are highly expressed during melanocyte and neural crest development (FIG. 5B) (Loftus et al., “Informatic Selection of a Neural Crest-melanocyte cDNA Set for Microarray Analysis,” Proc Natl Acad Sci USA 96:9277-80 (1999), which is hereby incorporated by reference in its entirety). To confirm the array data, levels of selected transcripts were measured by quantitative RT-PCR (FIG. 5C). To establish that the effects of WNT3A on gene expression are specific, it is shown that changes in gene expression are antagonized by β-catenin siRNA (FIG. 5D). The transcriptional profiling thus supports the hypothesis, evident from visual examination of cells (FIG. 5A), that activation of Wnt/β-catenin signaling by WNT3A promotes melanoma cells adopting characteristics of melanocyte differentiation.

Previous studies reporting increased expression of WNT5A observed in later-stage, more aggressive melanomas has focused on the potential role of noncanonical Wnt signaling on regulation of cell motility (Weeraratna et al., “Wnt5a Signaling Directly Affects Cell Motility and Invasion of Metastatic Melanoma,” Cancer Cell 1:279-88 (2002); Weeraratna et al., “Generation and Analysis of Melanoma SAGE Libraries: SAGE Advice on the Melanoma Transcriptome,” Oncogene 23: 2264-74 (2004); Da Forno et al., “WNT5A Expression Increases During Melanoma Progression and Correlates with Outcome,” Clin Cancer Res 14:5825-32 (2008); Bittner et al., “Molecular Classification of Cutaneous Malignant Melanoma by Gene Expression Profiling,” Nature 406:536-40 (2000); Witze et al., “Wnt5a Control of Cell Polarity and Directional Movement by Polarized Redistribution of Adhesion Receptors,” Science 320:365-9 (2008); Dissanayake et al., “The Wnt5A/protein Kinase C Pathway Mediates Motility in Melanoma Cells Via the Inhibition of Metastasis Suppressors and Initiation of an Epithelial to Mesenchymal Transition,” J Biol Chem 282:17259-71 (2007), which are hereby incorporated by reference in their entirety). In addition to regulating cell motility, WNT5A antagonizes Wnt/β-catenin signaling in developmental models and other contexts (Weidinger et al., “When Wnts Antagonize Wnts,” J Cell Biol 162:753-5 (2003), which is hereby incorporated by reference in its entirety). Consistent with this activity, it was shown that expression of WNT5A antagonizes the expression of genes regulated by WNT3A (FIG. 5C). These findings suggest that the antagonism of Wnt/β-catenin signaling by WNT5A may contribute to the loss of Wnt/β-catenin homeostasis seen with melanoma progression.

The exact role of Wnt/β-catenin signaling in melanoma remains controversial, although previous reports that the activation of Wnt/β-catenin signaling can promote proliferation in different cancers, including in cultured melanoma cells (Moon et al., “WNT and Beta-catenin Signalling: Diseases and Therapies,” Nat Rev Genet. 5:691-701 (2004); Widlund et al., “Beta-catenin-induced Melanoma Growth Requires the Downstream Target Microphthalmia-associated Transcription Factor,” J Cell Biol 158:1079-87 (2002), which are hereby incorporated by reference in their entirety), has led to speculation that this pathway may be involved in early aspects of melanoma formation and progression through regulation of tumor growth. In support of the idea that Wnt/β-catenin activation does not necessarily promote proliferation in melanocytic cells, a recent study found that forced transgenic expression of a stabilized constitutively-active β-catenin mutant (β-cat^{STA}) in mice did not increase proliferation of melanocytes or precursor melanoblasts (Delmas et al., “Beta-catenin Induces Immortalization of Melanocytes by Suppressing p16INK4a Expression and Cooperates with N-Ras in Melanoma Development,” Genes Dev 21:2923-35 (2007), which is hereby incorporated by reference in its entirety). This study also found that restricting the expression of β-cat^{STA} to melanocytes, in the absence of activated Nras, did not lead to any melanomas over a 2-year period (Delmas et al., “Beta-catenin Induces Immortalization of Melanocytes by Suppressing p16INK4a Expression and Cooperates with N-Ras in Melanoma Development,” Genes Dev 21:2923-35 (2007), which is hereby incorporated by reference in its entirety). However, the presence of both β-cat^{STA} and activated Nras led to melanomas with high penetrance and short latency, which suggests that in the context of this model, the activation of Wnt/β-catenin signaling can potentially promote melanoma formation (Delmas et al., “Beta-catenin Induces Immortalization of Melanocytes by Suppressing p16INK4a Expression and Cooperates with N-Ras in Melanoma Development,” Genes Dev 21:2923-35 (2007), which is hereby incorporated by reference in its entirety). Since activating mutations such as β-cat^{STA} are rare in melanoma (Omholt et al., “Cytoplasmic and Nuclear Accumulation of Beta-catenin is Rarely Caused by CTNNB1 Exon 3 Mutations in Cutaneous Malignant Melanoma,” Int J Cancer 92:839-42 (2001); Pollock et al., “Mutations in Exon 3 of the Beta-catenin Gene are Rare in Melanoma Cell Lines,” Melanoma Res 12:183-6 (2002); Reifenberger et al., “Molecular Genetic Analysis of Malignant Melanomas for Aberrations of the WNT Signaling Pathway Genes CTNNB1, APC, ICAT and BTRC,” Int J Cancer 100:549-56 (2002); Rubinfeld et al., “Stabilization of Beta-catenin by Genetic Defects in Melanoma Cell Lines,” Science 275:1790-2 (1997); Worm et al., “Genetic and Epigenetic Alterations of the APC Gene in Malignant Melanoma,” Oncogene 23:5215-26 (2004); Rimm et al., “Frequent Nuclear/cytoplasmic Localization of Beta-catenin Without Exon 3 Mutations in Malignant Melanoma,” Am J Pathol 154:325-9 (1999), which are hereby incorporated by reference in their entirety), and since the great majority of benign nevi do not progress to melanoma despite exhibiting nuclear β-catenin (Bachmann et al., “Importance of P-cadherin, Beta-catenin, and Wnt5a/Frizzled for Progression of Melanocytic Tumors and Prognosis in Cutaneous Melanoma,” Clin Cancer Res 11:8606-14 (2005), which is hereby incorporated by reference in its entirety), this model may not entirely recapitulate the role of Wnt/β-catenin signaling in human melanoma development and progression. Additionally, this model does not explain the observed increased survival (FIG. 1) seen in patients with tumors exhibiting higher levels of nuclear β-catenin (Bachmann et al., “Importance of P-cadherin, Beta-catenin, and Wnt5a/Frizzled for Progression of Melanocytic Tumors and Prognosis in Cutaneous Melanoma,” Clin Cancer Res 11:8606-14 (2005), which is hereby incorporated by reference in its entirety), which is accompanied by decreased, rather than increased, proliferation (FIGS. 4A and 4B).

Furthermore, transcriptional profiling reveals that activation of the Wnt/β-catenin pathway by WNT3A leads to upregulation of TRPM1, KIT, MET, and MLANA. These genes, which are associated with normal melanocyte differentiation, were recently identified as part of a transcriptional signature that is lost in aggressive melanomas compared to normal melanocytes (Ryu et al., “Comprehensive Expression Profiling of Tumor Cell Lines Identifies Molecular Signatures of Melanoma Progression,” PLoS ONE 2:e594 (2007), which is hereby incorporated by reference in its entirety), supporting a model in which the loss of Wnt/β-catenin-regulated genes is associated with both de-differentiation and melanoma progression. Consistent with this hypothesis, the loss of TRPM1 has been linked to decreased survival and an increased risk of metastasis (Duncan et al., “Melastatin Expression and Prognosis in Cutaneous Malignant Melanoma,” J Clin Oncol 19:568-76 (2001); Duncan et al., “Down-regulation of the Novel Gene Melastatin Correlates with Potential for Melanoma Metastasis,” Cancer Res 58:1515-20 (1998); Hammock et al., “Chromogenic In Situ Hybridization Analysis of Melastatin mRNA Expression in Melanomas from American Joint Committee on Cancer Stage I and II Patients with Recurrent Melanoma,” J Cutan Pathol 33:599-607 (2006), which are hereby incorporated by reference in their entirety), and the ability of Wnt/β-catenin signaling to rescue the transcriptional regulation of TRPM1 and other genes lost with melanoma progression provides further evidence that loss of Wnt/β-catenin homeostasis may play a direct role in melanoma progression. The results also suggest that WNT5A, which is expressed at higher levels with melanoma progression (Weeraratna et al., “Wnt5a Signaling Directly Affects Cell Motility and Invasion of Metastatic Melanoma,” Cancer Cell 1:279-88 (2002); Weeraratna et al., “Generation and Analysis of Melanoma SAGE Libraries: SAGE Advice on the Melanoma Transcriptome,” Oncogene 23: 2264-74 (2004); Da Formo et al., “WNT5A Expression Increases During Melanoma Progression and Correlates with Outcome,” Clin Cancer Res 14:5825-32 (2008); Bittner et al., “Molecular Classification of Cutaneous Malignant Melanoma by Gene Expression Profiling,” Nature 406:536-40 (2000), which are hereby incorporated by reference in their entirety), may directly contribute to this dysregulation by antagonizing Wnt/β-catenin transcriptional targets.

Given the established role of Wnt/β-catenin signaling as a main regulator of cell fate in the melanocytic lineage, the results invite speculation that WNT3A may be altering the differentiation of melanoma cells in this model. This hypothesis is supported by observations that melanoma cells expressing WNT3A exhibit properties suggestive of more highly differentiated melanocytic cells, including: 1) a high degree of pigmentation; 2) alterations in cell cycle leading to decreased proliferation; 3) upregulation of melanocytic genes; and 4) formation of smaller tumors in explant studies. The concept of manipulating the differentiation of tumor cells in melanoma and other cancers is not new (Postovit et al., “Influence of the Microenvironment on Melanoma Cell Fate Determination and Phenotype,” Cancer Res 66:7833-6 (2006), which is hereby incorporated by reference in its entirety), and, in fact, recent research has focused on markers that may better identify so-called melanoma stem cells, or tumor initiating cells, that would be the ideal targets for such a therapeutic strategy (Zabierowski et al., “Melanoma Stem Cells: the Dark Seed of Melanoma,”J Clin Oncol 26:2890-4 (2008), which is hereby incorporated by reference in its entirety). The forced differentiation of these tumor initiating populations would ideally promote cell fates that are more benign (i.e. slower growing, or less metastatic) or alternatively more treatable.

### Example 4

### High-throughput Screening Identifies Drugs that are Stand-alone and Synergistic Activators of Wnt/β-catenin Signaling in Melanoma Cells

A high-throughput screen of over 30,000 compounds was conducted using an optimized Wnt/β-catenin-activated reporter (Biechele, T. L., et al., “Assaying Beta-catenin/TCF Transcription with Beta-catenin/TCF Transcription-based Reporter Constructs,” Methods Mol Biol 468: 99-110 (2008), which is hereby incorporated by reference in its entirety) expressed in HEK293 cells as well as in a cultured hippocampal cell line. This screen identified over 250 compounds that could activate Wnt/β-catenin signaling either alone or in the presence of WNT3A (as conditioned media). Several of these compounds were subsequently validated in A2058 melanoma cells, as shown in FIG. 10. This data, which encompasses a subset of the compounds identified in the screen, illustrates that small molecules such as lovastatin, kenpaullone, riluzole, L744,832 and 13-cis retinoic acid (13-cis RA) exhibit the ability to activate Wnt/β-catenin signaling on their own. Other compounds such as forskolin and 6-mercaptopurine (6-MP) exhibit no activation of Wnt/β-catenin signaling on their own, but are able to synergize with WNT3A to increase Wnt/13-catenin signaling. Interestingly, the drugs that are stand-alone activators also exhibit significant synergy with WNT3A.

### Example 5

### Riluzole Activates Wnt/β-catenin Signaling in Melanoma Cells

The aminobenzothiazole drug riluzole (brand name Rilutek) was identified in a screen as both an activator and synergizer for Wnt/β-catenin signaling. Data reduction based on scoring drugs that could both activate and synergize with Wnt/β-catenin signaling found that riluzole in two unique preparations represented two of the top six hits in this screen (FIG. 11). This drug is FDA-approved for the treatment of amyotrophic lateral sclerosis based on a proposed mechanism involving the inhibition of glutamate signaling through the protein GRM1. Riluzole was able to activate a second independent Wnt/β-catenin reporter and enhance the transcription of Wnt/β-catenin target genes in murine melanoma cells in a dose dependent manner (FIG. 11), validating the initial identification of this drug as a modifier of Wnt/β-catenin signaling in the screen.

### Example 6

### Riluzole Mimics Effects of Wnt/β-catenin Activation in Melanoma Cells

Previously, it was demonstrated that activation of Wnt/β-catenin signaling increased pigmentation and decreased proliferation in melanoma cells, which is accompanied by changes in the regulation of Wnt target genes (Chien, A. J., et al., “Activated Wnt/beta-catenin Signaling in Melanoma is Associated with Decreased Proliferation in Patient Tumors and a Murine Melanoma Model,” Proc Natl Acad Sci USA 106: 1193-1198 (2009), which is hereby incorporated by reference in its entirety.) In murine B16 melanoma cells, riluzole was able to induce pigmentation, upregulate Wnt target genes, inhibit proliferation in culture, and decrease metastasis in vivo (FIG. 12). In human melanoma cells, riluzole exhibited dose-dependent activation of the Wnt reporter and also inhibited the proliferation of virtually all human melanoma lines tested with the exception of 501 MEL, which already has active Wnt/β-catenin signaling secondary to an activating mutation in O-catenin.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1-8. (canceled)

9. A method of improving survival of a melanoma patient, decreasing metastases in a melanoma patient, decreasing proliferation of cancer cells in a melanoma patient, decreasing melanoma recurrence in a melanoma patient, and/or decreasing tumor size in a melanoma patient, said method comprising:
selecting a melanoma patient based on their level of Wnt/β-catenin and
treating the selected melanoma patient with a dose of an activator or synergizer of Wnt/βcatenin based on the patient's level of Wnt/β-catenin, said treating being carried out under conditions effective, respectively, to improve survival of the selected melanoma patient, to decrease metastases in the selected melanoma patient, to decrease proliferation of cancer cells in the selected melanoma patient, to decrease melanoma recurrence in the selected melanoma patient, and/or to decrease tumor size in the selected melanoma patient.

10. The method of claim 9, wherein said treating is carried out with an activator of Wnt/β-catenin.

11. The method of claim 10, wherein the activator of Wnt/β-catenin is a glycogen synthase kinase-3 (GSK-3) inhibitor.

12. The method of claim 10, wherein the activator of Wnt/β-catenin is selected from the group consisting of riluzole, flunarizine, 6-bromoindirubin-3′-oxime (BIO), lithium ions, insulin, insulin-like growth factor (IGF-1), epidermal growth factor (EGF), CHIR-911, CHIR-837, CHIR-98014, CHIR-99021, CHIR-99030, and CHIR-98023.

13. The method of claim 9, wherein said treating is carried out with a synergizer of Wnt/β-catenin.

14. The method of claim 9, wherein the dose of activator or synergizer increases the level of Wnt/β-catenin in the selected melanoma patient.

15. The method of claim 9 further comprising:
modifying said treating as indicated by the results of a test determining the level of an indicator of Wnt/β-catenin activation following said treating.

16. The method of claim 9, wherein said selecting comprises:
identifying as a selected melanoma patient a patient with elevated levels of Wnt/β-catenin as determined by the results of a test comprising:
determining the level of an indicator of Wnt/β-catenin activation in a biological sample from a patient diagnosed with melanoma;
comparing the patients' level of the indicator of Wnt/β-catenin activation against a standard level of the indicator Wnt/β-catenin correlated, respectively, to survival of melanoma patients, decreased metastases in melanoma patients, decreased proliferation of cancer cells in melanoma patients, decreased melanoma recurrence in melanoma patients, and/or decreased tumor size in melanoma patients.

17. The method of claim 9, wherein said treating is carried out by intravesicular, intrathecal, parenteral, topical, intravenous, oral, inhalant, subcutaneous, intraarterial, intracranial, intraperitoneal, intranasal, or intramuscular administration.

18. The method of claim 9, wherein survival of the melanoma patient is improved.

19. The method of claim 9, wherein there is a decrease in metastasis, proliferation of cancer cells, recurrence of melanoma, and/or tumor size in the melanoma patient.

20-22. (canceled)

23. The method of claim 9, wherein patient response to said treating is monitored by said measuring of Wnt/β-catenin activation.

24. A method of predicting the likelihood of survival of a melanoma patient, said method comprising:
determining the level of an indicator of Wnt/β-catenin activation in a biological sample from
a patient diagnosed with melanoma;
comparing the level of the indicator of Wnt/β-catenin activation in the sample against a standard level of the indicator of Wnt/β-catenin activation correlated to survival of melanoma; and
determining a patient's prospects for survival of melanoma based on said comparing

25. The method of claim 24, wherein the level of the indicator is determined using one or more methods from the group consisting of:
quantitative PCR; immunoassays; ELISA; radioimmunoassay; immunoradiometric assay,
Western blotting; PCR; immunohistochemistry; and microarray analysis.

26. The method of claim 24, wherein the melanoma patient is a human.

27. The method of claim 24, wherein the biological sample is selected from the group consisting of surgically-excised primary tumors and metastatic lesions.

28. The method of claim 24, wherein the level of the indicator is the expression level of a gene that is a marker for activation of the Wnt/β-catenin pathway.

29. The method of claim 28, wherein the expression level of the gene that is the marker for activation of the Wnt/β-catenin pathway is determined by quantitative PCR.

30. The method of claim 28, wherein the gene is AXIN2.

31. The method of claim 24, wherein the indicator is β-catenin assessed by a quantitative assay.

32. The method of claim 31, wherein the level of the indicator is determined with an antibody which recognizes β-catenin.
